SEQUENCE LISTING (4 sequences)

SEQ ID NO: 1 - DNA, 17 bp, Artificial Sequence
  T7-linked primers for PCR amplification
acctgtggacgccaagg

SEQ ID NO: 2 - DNA, 19 bp, Artificial Sequence
  T7-linked primers for PCR amplification
aaaagaagtcgacggcttc

SEQ ID NO: 3 - DNA, 20 bp, Artificial Sequence
  T7-linked primers for PCR amplification
gataggattgcggatgagga

SEQ ID NO: 4 - DNA, 20 bp, Artificial Sequence
  T7-linked primers for PCR amplification
tccaatgaagaagaatcggg

(19) United States
(12) Patent Application Publication
(10) Pub. No.: US 2010/0267626 A1
(43) Pub. Date: Oct. 21, 2010

(54) METHODS AND COMPOSITIONS FOR MEASURING WNT ACTIVATION AND FOR TREATING WNT-RELATED CANCERS

(75) Inventors: Atwood Cheung, Cambridge, MA (US); Feng Cong, Cambridge, MA (US); Shih-Min Huang, Cambridge, MA (US)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: Novartis AG

(21) Appl. No.: 12/741,114

(22) PCT Filed: Nov. 5, 2008
(86) PCT No.: PCT/EP08/64987

§ 371 Date: May 3, 2010

Related U.S. Application Data
(60) Provisional application No. 60/985,454, filed on Nov. 5, 2007.

(51) Int. Cl. A61K 38/00; C12Q 1/68; C12Q 1/02; A61P 3/04; A61P 3/10; A61P 13/12; A61P 19/02; A61P 25/00; A61P 35/00; A61K 31/7088

(52) U.S. Cl. 514/4.8; 435/6; 435/29; 514/1.1; 514/6.9; 514/15.4; 514/16.8; 514/17.7; 514/19.3; 514/44 R

(57) ABSTRACT

The present application describes methods of regulating or modulating (e.g., antagonizing or inhibiting) Wnt signaling by administering Axin stabilizers. The application also describes methods of using Axin stabilizers described herein for the treatment, diagnosis, prevention, and/or amelioration of Wnt signaling-related disorders.

### BACKGROUND OF THE INVENTION

The Wnt gene family encodes a large class of secreted proteins related to the Int1/Wnt1 proto-oncogene and Drosophila wingless (“Wg”), a Drosophila Wnt1 homologue (Cadigan et al. (1997) Genes & Development 11:3286-3305). Wnts are expressed in a variety of tissues and organs and are required for many developmental processes, including segmentation in Drosophila; endoderm development in C. elegans; and establishment of limb polarity, neural crest differentiation, kidney morphogenesis, sex determination, and brain development in mammals (Parr, et al. (1994) Curr. Opinion Genetics & Devel. 4:523-528). The Wnt pathway is a master regulator in animal development, both during embryogenesis and in the mature organism (Eastman, et al. (1999) Curr Opin Cell Biol 11: 233-240; Peifer, et al. (2000) Science 287: 1606-1609).

Wnt signals are transduced by the Frizzled (“Fz”) family of seven transmembrane domain receptors (Bhanot et al. (1996) Nature 382:225-230). Wnt ligands bind to Fzd, and in so doing, activate the cytoplasmic protein Dishevelled (Dvl-1, 2 and 3 in humans and mice) (Boutros, et al. (1999) Mech Dev 83: 27-37) and phosphorylate LRP5/6. A signal is thereby generated which prevents the phosphorylation and degradation of Armadillo/β(beta)-catenin, in turn leading to the stabilization of β-catenin (Perrimon (1994) Cell 76:781-784). This stabilization is occasioned by Dvl's association with axin (Zeng et al. (1997) Cell 90:181-192), a scaffolding protein that brings various proteins together, including GSK3, APC, CK1, and β-catenin, to form the β-catenin destruction complex.

Glycogen synthase kinase 3 (GSK3, known as shaggy in Drosophila), the tumor suppressor gene product APC (adenomatous polyposis coli) (Gumbiner (1997) Curr. Biol. 7:R443-436), and Axin, are all negative regulators of the Wnt pathway. In the absence of a Wnt ligand, these proteins form a complex and promote phosphorylation and degradation of β-catenin, whereas Wnt signaling inactivates the complex and prevents β-catenin degradation. Stabilized β-catenin translocates to the nucleus as a result, where it binds TCF (T cell factor) transcription factors (also known as lymphoid enhancer-binding factor-1 (LEF1)) and serves as a coactivator of TCF/LEF-induced transcription (Bienz, et al. (2000) Cell 103: 311-320; Polakis, et al. (2000) Genes Dev 14: 1837-1851).

Aberrant Wnt pathway activation, through the stabilization of β-catenin, plays a central role in tumorigenesis for many colorectal carcinomas. It is estimated that 80% of colorectal carcinomas (CRCs) harbor inactivating mutations in the tumor repressor APC, which allows for uninterrupted Wnt signaling. Furthermore, there is a growing body of evidence that suggests that Wnt-pathway activation may be involved in melanoma, breast, liver, lung, and gastric cancers. There is a long-recognized connection between Wnts, normal development, and cancer, a connection further established with the identification the c-Myc proto-oncogene as a target of Wnt signaling (He et al. (1998) Science 281:1509-3512).

Furthermore, other disorders are associated with aberrant Wnt signaling, including but not limited to osteoporosis, osteoarthritis, polycystic kidney disease, diabetes, schizophrenia, vascular disease, cardiac disease, non-oncogenic proliferative diseases, and neurodegenerative diseases such as Alzheimer's disease.

The current paradigm for developing therapies for Wnt signaling-related disorders, such as colorectal cancer, relies on targeting β-cat or Wnt pathway components downstream of β-cat. Recent studies, however, suggest that autocrine Wnt signaling mediated by Wnt receptor Frizzled and LRP5/6 may play key roles in regulating tumor growth and survival. A need exists for agents and methods that inhibit Wnt signal transduction activity by modulating activation of the Wnt pathway along other critical junctures, thereby treating, diagnosing, preventing, and/or ameliorating Wnt signaling-related disorders.

### SUMMARY OF THE INVENTION

The present invention provides methods of diagnosing, ameliorating the symptoms of, protecting against, and treating Wnt signaling-related disorders (e.g., colorectal cancer), e.g., through use of agents that modulate the protein stability and/or levels of Axin (e.g., small molecules (including, e.g., the compounds of the invention), inhibitory nucleic acids, fusion proteins, etc.). In some embodiments, said agents modulate Tankyrase (TNKS), e.g., by modulating the TNKS catalytic activity.

Said methods may include administering to a subject in need thereof an effective amount of a modulator of Wnt pathway signal transduction, e.g., an Axin stabilizer and/or a TNKS modulator (e.g., small molecule (including, e.g., a compound of the invention), inhibitory nucleic acid, fusion protein, or any combination thereof), and a pharmaceutically acceptable carrier.

Said methods can be used at the cellular level, e.g., to treat epithelial cells having a Wnt receptor. For instance, the subject method can be used in treating or preventing basal cell carcinoma or other Wnt signaling-related disorders (e.g., those characterized by aberrant cell proliferation). The subject methods can also be used to prevent cellular proliferation, aberrant or otherwise, by inhibiting or agonizing the inhibition of Wnt signal transduction. Said methods can also be used for both human and animal subjects.

The present invention also provides methods of modulating Wnt pathway signal transduction, e.g., through use of Axin stabilizers, e.g., through use of TNKS modulators (e.g., small molecules, inhibitory nucleic acids, fusion proteins, etc.). In one embodiment, the methods of the present invention include inhibiting or agonizing the inhibition of Wnt pathway signal transduction, e.g., through use of Axin stabilizers, e.g., through use of TNKS modulators (e.g., small molecules, inhibitory nucleic acids, fusion proteins, etc.).

Said methods of inhibiting or agonizing the inhibition of Wnt pathway signal transduction can be employed, e.g., in the regulation of repair and/or functional performance of a wide range of cells, tissues and organs, including normal cells, tissues, and organs. Non-limiting examples include regulation of neural tissues, bone and cartilage formation and repair, regulation of spermatogenesis, regulation of smooth muscle, regulation of lung, liver and other organs arising from the primitive gut, regulation of hematopoietic function, regulation of skin and hair growth, etc. The methods of the present invention can be performed in vitro or in vivo.

The present invention also provides methods of identifying and testing agonists and antagonists of Wnt pathway signal transduction, and methods of identifying and testing agonists and antagonists of Wnt pathway members (e.g., Axin, TNKS). The present discovery of inhibiting Wnt pathway signal transduction through the stabilization and raising of Axin protein levels, and through the inhibition of TNKS, is useful for identifying agents that will enhance or interfere with this stabilization, and thereby with Wnt pathway signaling, in vitro or in vivo. The present discovery is thereby also useful for discovering agents that can inhibit TNKS catalytic activity, and can thereby be used to treat disorders associated aberrant and pathological Wnt signaling that can result from, e.g., the failure of Axin to stabilize and form its β-catenin destruction complex (and the resulting modulation of Wnt pathway signal transduction).

In one embodiment, a method of identifying an agent capable of modulating Wnt pathway signal transduction, comprises: a) contacting a biological sample in which the Wnt signaling pathway is active in the presence and absence of a test agent under conditions permitting Wnt signaling and in which TNKS protein levels can be measured; and b) measuring the levels of TNKS protein in both the presence and absence of said test agent, wherein (i) a decrease in TNKS protein levels or stability in the presence of the test agent, relative to the absence of the test agent, identifies the test agent as an antagonist of Wnt pathway signal transduction, and wherein (ii) an increase in TNKS protein levels or stability in the presence of the test agent, relative to the absence of the test agent, identifies the test agent as an agonist of Wnt pathway signal transduction.

In one embodiment, the agent can be a small molecule. In another embodiment, the agent can be an inhibitory nucleic acid (e.g., an anti-TNKS1 or -TNKS2 siRNA). In another embodiment, the agent can be a fusion protein (e.g., an inhibitory fusion protein against TNKS).

The present invention includes a method for screening compounds useful for the treatment of Wnt signaling-related disorders (e.g., colorectal cancer), comprising contacting a cell exhibiting Wnt pathway signal transduction with a test agent and detecting a change in TNKS protein levels, or in Axin protein levels and/or Axin stabilization.

In one embodiment, a method of identifying an agent useful for the treatment of Wnt signaling-related disorders, comprises: a) contacting a biological sample in which the Wnt signaling pathway is active in the presence and absence of a test agent under conditions permitting Wnt signaling and in which TNKS protein or stability levels can be measured; and b) measuring the levels of TNKS protein in both the presence and absence of said test agent, wherein (i) a decrease in TNKS protein levels or stability in the presence of the test agent, relative to the absence of the test agent, identifies the test agent as useful for treating disorders associated with aberrant upregulation of Wnt signaling, and wherein (ii) an increase in TNKS protein levels or stability in the presence of the test agent, relative to the absence of the test agent, identifies the test agent as useful for treating disorders associated with aberrant downregulation of Wnt signaling.

In one embodiment, the agent can be a small molecule. In another embodiment, the agent can be an inhibitory nucleic acid (e.g., an anti-TNKS1 or -TNKS2 siRNA). In another embodiment, the agent can be a fusion protein (e.g., an inhibitory fusion protein against TNKS).

In one embodiment, a method for identifying agents useful for the treatment of Wnt signaling-related disorders comprises contacting a cell in which the Wnt signaling pathway is active with a test agent and detecting a change in the TNKS protein levels or stability.

In the screening methods of the invention, an increase in Axin protein levels or stability is measured by a decrease in total β-catenin levels, an increase in phospho-β-catenin levels, an increase in Axin protein levels, or increased formation of the Axin-GSK3 complex. A decrease in Axin protein levels or stability is measured by an increase in total β-catenin levels, a decrease in phospho-β-catenin levels, a decrease in Axin protein levels, or decreased formation of the Axin-GSK3 complex.

Other screening methods of the present invention include methods of identifying agents capable of modulating Wnt pathway signal transduction, and methods of identifying agents capable of inhibiting the catalytic activity of Tankyrase (TNKS). Said agents can be at least a small molecule, an inhibitory nucleic acid (e.g., an anti-TNKS1 or -TNKS2 siRNA), or a fusion protein (e.g., an inhibitory fusion protein against TNKS)

The present invention includes pharmaceutical preparations comprising, as an active ingredient, a Wnt antagonist (e.g., an Axin stabilizer and/or a TNKS antagonist), such as described herein, formulated in an amount sufficient to (i) inhibit, in vivo, cellular proliferation or other biological consequences of Wnt aberrant expression (e.g., in cancers characterized by constitutive Wnt signaling); and (ii) to diagnose, ameliorate the symptoms of, protect against, or treat Wnt signaling-related disorders. Said preparation can, e.g., include a compound, an inhibitory nucleic acid, or a fusion protein according to any embodiment of the present invention, or any combination thereof, in a pharmaceutically acceptable carrier.

In another embodiment, a method for inhibiting growth of a tumor cell is provided, which involves administering to a subject in need thereof an effective amount of a modulator of Wnt pathway signal transduction, e.g., an Axin stabilizer and/or a TNKS antagonist (e.g., small molecule (including, e.g., a compound of the invention), an inhibitory nucleic acid, fusion protein, etc., or any combination thereof), and a pharmaceutically acceptable carrier.

Further provided is a method for inducing apoptosis in a tumor cell, which includes administering to a subject in need thereof an effective amount of a modulator of Wnt pathway signal transduction, e.g., an Axin stabilizer and/or a TNKS antagonist (e.g., small molecule (including, e.g., a compound of the invention), an inhibitory nucleic acid, fusion protein, etc., or any combination thereof), and a pharmaceutically acceptable carrier.

The present invention includes methods for identifying or predicting the predisposition or likelihood of subjects afflicted with Wnt signaling-related disorders (e.g., colorectal cancer) to benefit from a treatment regiment that includes “TNKS inhibitor,” “Axin stabilizers,” or the like (including, e.g., the compounds of the invention, or fusion proteins or inhibitory nucleotides capable of inhibiting the catalytic activity of TNKS).

### BRIEF DESCRIPTION OF THE DRAWINGS

FIG. 1. XAV939 inhibits Wnt/β-catenin signaling by increasing Axin protein level.

FIG. 1A. XAV939 specifically inhibits STF activity in HEK293 cells. HEK293 STF, CRE, NFκB, and CAGA12 reporter cell lines were activated with Wnt3A conditioned medium, Forskolin, TNFα, and TGFβ, respectively, and treated with 12-point dilutions of XAV939 or LDW643 (an inactive analog, and negative control, of compound for XAV939). The corresponding reporter activity for each compound dilution was normalized to DMSO and expressed as a percentage of the reporter activity in DMSO.

FIG. 1B. XAV939 reduces Wnt3A stabilized β-catenin levels. HEK293 cells were stimulated with Wnt3A conditioned media for the indicated length of time, in the presence of DMSO or 1 μM XAV939. Cell lysates were fractionated and immunoblotted for cytosolic β-catenin.

FIG. 1C. XAV939 inhibits STF activity in APC-deficient SW480 cells. SW480-STF and SW480-STF-TCF3P16 cells were treated with 12-point dilutions of XAV939 or LDW643 and assayed for luciferase activity. Overexpression of TCF3VP16 fusion protein largely bypassed the requirement of β-catenin on STF activity (data not shown). Similar to b, The corresponding reporter activity for each compound dilution point was normalized to the reporter activity in DMSO and presented as a percentage.

FIG. 1D. XAV939 decreases the abundance of β-catenin and increases the abundance of Axin and phospho-β-catenin. SW480 cells were treated overnight with 1 μM XAV939 or LDW643, fractionated for cytosolic proteins, and immunoblotted with the indicated antibodies.

FIG. 1E. The effect of XAV939 on β-catenin is Axin dependent. SW480 cells transfected with both Axin1 and Axin2 siRNAs or control pGL2 siRNA were treated in the presence or absence of 3 μM XAV939. Cytosolic lysates were then isolated and immunoblotted with the indicated antibodies.

FIG. 2. Identification of the cellular efficacy targets.

FIG. 2A. Immunoblot analysis for TNKS1/2, PARP1, PARP2 on lysates from a dose response compound competition experiment with ascending doses, ranging from 1 nM to 100 μM in steps of 10-fold increments, of the active compound XAV939 and the inactive analog LDW643.

FIG. 2B. XAV939 directly binds PARP domain of TNKS1 and TNKS2 with higher affinity. GST-TNKS1 and TNKS2 were incubated with XAV939 conjugated to Cy5. Raw mP [1000×(S−G*P/S+G*P)] data were exported and analyzed with a one-site total binding saturation algorithm.

FIG. 3. Tankyrase modulates Axin protein level

FIGS. 3A, B. Simultaneous depletion of TNKS1 and TNKS2 phenocopies XAV939 by increasing Axin protein levels and decreasing β-catenin protein level. SW480 cells were transfected with siRNA singletons against PARP1, PARP2, TNKS1, and TNKS2 in the indicated combinations. For both TNKS 1 and 2, two independent siRNAs generated from unique target sequences, labeled A and B, were utilized in the experiment. Cytosolic proteins were harvested 48 hours after transfection and analyzed by the indicated antibodies.

FIG. 3C. Depletion of TNKS1 and TNKS2 increases the protein level of Axin1 and blocks Wnt3a-induced β-catenin accumulation. HEK293 cells were transfected with individual siRNA against TNKS1 or TNKS2 in the indicated combinations. Upper panel, the expression of Axin1 was analyzed by immunoblotting 48 hours post transfection. Lower panel, cells were stimulated for 6 hours with Wnt3A conditioned media 48 hours post-transfection. Cytosolic β-catenin was then isolated and measured by immunoblotting.

FIG. 3D. Depletion of TNKS1 and TNKS2 specifically inhibits Wnt reporter. HEK293 CRE and STF reporter cell lines were transfected with indicated siRNAs, stimulated with Forskolin and Wnt 3A conditioned media, respectively, and measured for luciferase activity. Data is normalized against pGL2 control siRNA and displayed as a percent of inhibition.

FIG. 3E. Knockdown of TNKS increases the protein level of Axin in Drosphila S2 cells. S2 cells stably expressing DAxin-3xHA were incubated with control dsRNA (White) or dsRNA against Drosophila TNKS. The protein and mRNA levels of DAxin-3xHA were detected by immunoblotting (left panel) and qPCR (right panel).

FIG. 3F. Knock-down TNKS specifically inhibits Wnt reporter in Drosophila S2 cells. S2 were treated with the indicated dsRNA, transiently transfected with the Wnt (LEF-Luc), BMP (BRE-Luc) and JAK/SAT (Draf-Luc) reporters, stimulated with the appropriate ligands (Wingless conditioned medium, BMP2, and UPD conditioned medium), and assayed for luciferase activity.

FIG. 3G. Wild-type but not catalytically inactive TNKS2 rescues TNKS1/2 siRNA induced accumulation of Axin1. HEK293 cells stably expressing inducible siRNA resistant and Flag-tagged wildtype (WT) or catalytically inactive (M1054V) were transfected with siRNAs against TNKS1 and TNKS2. After doxycyclin (DOX) induced expression of exogenous TNKS2, cell lysates were harvested and analyzed by immunoblotting.

FIG. 3H. XAV939 inhibits autoparsylation of TNKS. 1 uM protein (GST-TNKS2-SAM-PARP1) was mixed with 5 uM biotin-NAD in with DMSO or 2 uM of XAV939 or LDW643 at 30° C. The samples were analyzed by SDS-PAGE and western blotting.

FIG. 4. Tankyrase physically and functionally interacts with Axin

FIG. 4A. Co-immunoprecipitation of endogenous Axin2 and Tnks. SW480 cells were transfected with control siRNA or Axin2 siRNA, and cell lysates were immunoprecipitated with anti-Axin2 antibody or IgG. Immunoprecipitates were resolved by SDS-PAGE and blotted with the indicated antibodies.

FIG. 4B. Mapping the TNKS binding domain of Axin1 using the yeast two-hybrid assay. Left panel, schematic depicting the Axin1 protein fragments used to bind Tnks in the yeast two-hybrid assay. Right panel, table summary of Axin1 protein fragment binding strength to TNKS in the yeast two-hybrid assay (+strong binding, +/−weak binding, −no binding). GSK3β, a known Axin1 binder, was used as a control. Note that the N88 fragment retains partial self-activation activity.

FIG. 4C. Cell lysates of HEK293 cells overexpressing Flag-TNKS1 were incubated with the indicated GST fusion proteins and precipitated before being immunoblotted and analyzed with the indicated antibodies. GST-AxinN consists of the amino-terminal 87 amino acid residues of Axin1 fused to GST.

FIG. 4D. Co-immunoprecipitation of Axin1 proteins and TNKS1. Cell lysates of HEK293 cells transfected with the indicated constructs were immunoprecipitated with anti-Flag antibodies and analyzed by immunoblotting.

FIG. 4E. The TNKS binding domain is required for XAV939-induced Axin1 protein accumulation. SW480 cell lines stably expressing the indicated GFP-Axin fusion constructs were established by retroviral infection, and treated with XAV939 overnight. Total cell lysates were harvested and analyzed by immunoblotting.

FIG. 4F. Overexpression of the amino terminal fragment of Axin1 leads to accumulation of endogenous Axin1. HEK293 cell lines expressing inducible GFP-Axin1N (a.a 1-87) were generated. Expression of GFP-Axin1N was induced by treating cells with doxocyclin (DOX) for 24 hours. Total cell lysates were harvested and analyzed by immunoblotting.

FIG. 4G. Various TNKS1 fragments were tested for their binding to Axin1 in a yeast two hybrid assay and for their effect on the STF reporter when overexpressed in HEK293 cells. Left panel, schematic of TNKS1 constructs and a summary of their ability to bind Axin1. The Axin1 binding activity of these constructs to Axin1 is indicated in the middle column, under “β-Gal Assay”. Right panel, the effect of the TNKS1 constructs on the STF reporter. TNKS1 constructs were transiently transfected into HEK293 STF reporter cells and assayed for luciferase activity 48 hrs post-transfection. (IP: immunoprecipitation, TCL: total cell lysates)

FIG. 5. XAV939 stabilize Axin protein level and inhibit Axin ubiquitylation

FIG. 5A. Axin is stabilized by XAV939. SW480 cells were treated with either DMSO or 1 μM XAV939 for 2 hrs prior to pulse-chase analysis, as described in Materials and methods. Cell lysates were prepared with RIPA buffer, immunoprecipitated with anti-Axin2 antibody, resolved by SDS-PAGE, and then analyzed with a PhosphoImager.

FIG. 5B. In vitro PARsylation of an Axin1 fragment by TNKS2. Recombinant TNKS2 and GST-Axin1 (a.a. 1-280) were incubated with biotin-NAD⁺. The reaction was carried out with or without presence of XAV939, resolved by SDS-PAGE, and probed with streptavidin-AlexaFluor680.

FIG. 5C. Axin ubiquitylation is inhibited by XAV939. SW480 cells were pretreated with 1 μM of XAV939 for 4 hours and subsequently treated with 20 μM of MG132 for an additional 2 hours. Cell lysates were harvested with RIPA buffer, immunoprecipitated with control IgG or anti-Ubiquitin antibody, immunoblotted and analyzed with the indicated antibodies. The position that Axin1 migrates is labeled with an arrow. Slow migrating poly-ubiquitinylated-Axin1 conjugates are indicated.

FIG. 5D. In vivo PARsylation of Axin1. SW480 cells stably expressing GFP-Axin1 under control of the metallothionein promoter were incubated with Cu²⁺ overnight to induce expression of GFP-Axin1. Cells were treated with XAV939 for an additional 6 hours. Lysates were harvested with RIPA buffer containing PARG inhibitor ADP-HPD (5 μM) and PARP1 inhibitor PJ34 (80 μM), immunoprecipitated with GFP antibody and analyzed by immunoblotting.

FIG. 5E. Post-translational modification of Axin2 in a compound wash-off experiment. SW480 cells were treated with 1 μM XAV939 overnight, washed with fresh medium to remove XAV939, and then incubated with medium supplemented with the indicated compound for 1 hour. Cell lysates were harvested with RIPA buffer, immunoprecipitated with anti-Axin2 antibody, and analyzed by immunoblotting. The position that Axin2 migrates is indicated by the arrows. (IP: immunoprecipitation, TCL: total cell lysates)

FIG. 6. XAV939 inhibits DLD1 colony formation in a Axin-dependent manner

FIG. 6A. XAV939 inhibits colony formation of DLD1, but not RKO, cells. DLD1 and RKO cells were seeded at 500 cells/well in a 6-well plate in medium containing 0.5% serum and indicated compounds, and replenished with fresh medium every two days. Colonies were visualized by crystal violet staining.

FIG. 6B. The effect of XAV939 on DLD1 colony formation is Axin-dependent. DLD1 cells were transfected with siRNAs against Axin1 and Axin2, and seeded at 1000 cells/well in a 6-well plate. The compound treatment was carried out as described in 6A.

FIG. 7 depicts the ability of a compound of invention to inhibit the growth of multiple cancer models characterized by activated Wnt signaling, including cancers that have loss-of-function APC mutations, cancers that have gain-of-function β-catenin activating mutations, and/or cancers with activated Wnt signaling demonstrated by the high expression levels of β-catenin target gene Axin2. Three representative examples are shown in the Figure, including a colorectal cancer cell line SW403 that has an APC mutation, a colorectal cancer cell line HuTu-80 that has a β-catenin mutation, and a gastric cancer cell line NCI-N87 that has high level of Axin2 gene expression demonstrated by both quantitative PCR and by gene expression microarray.

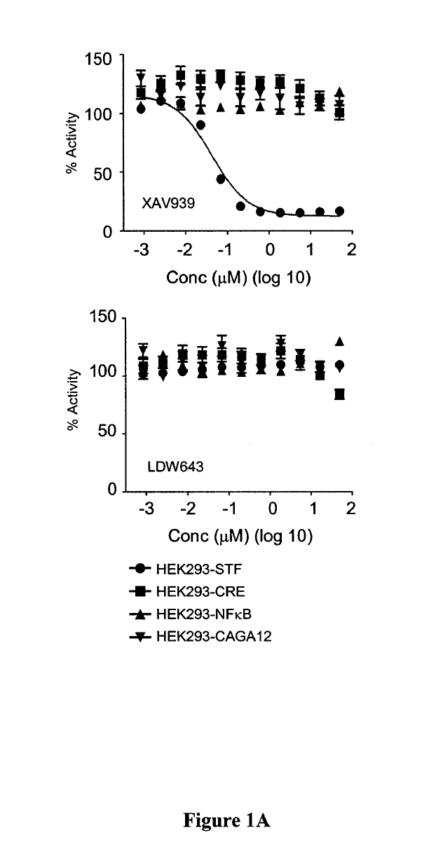

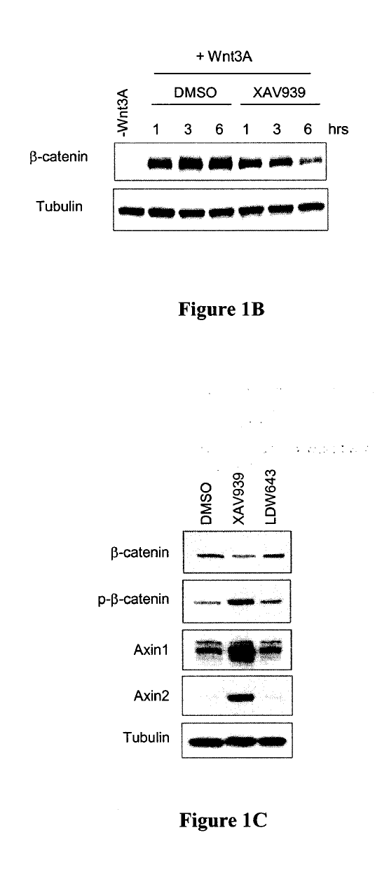

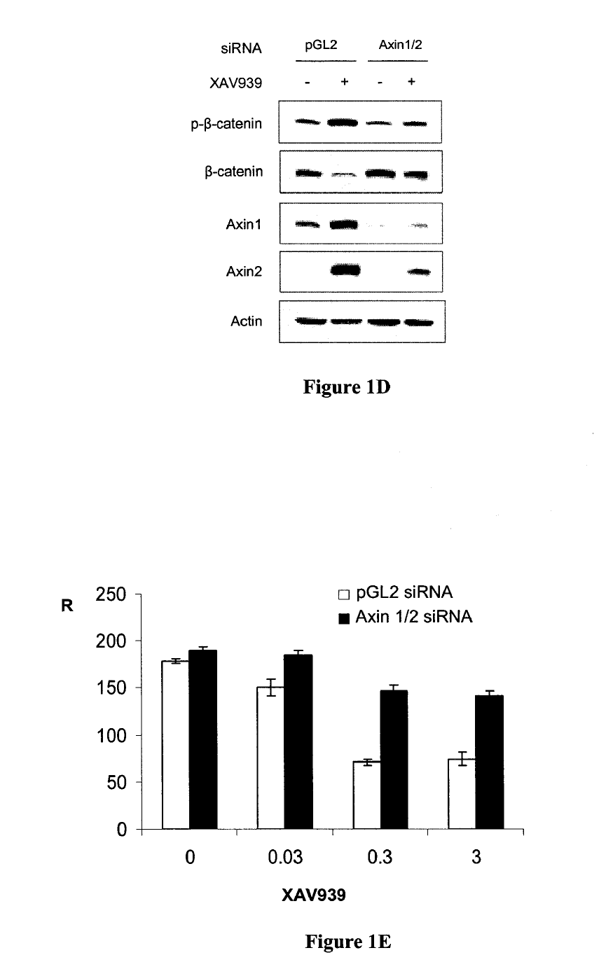

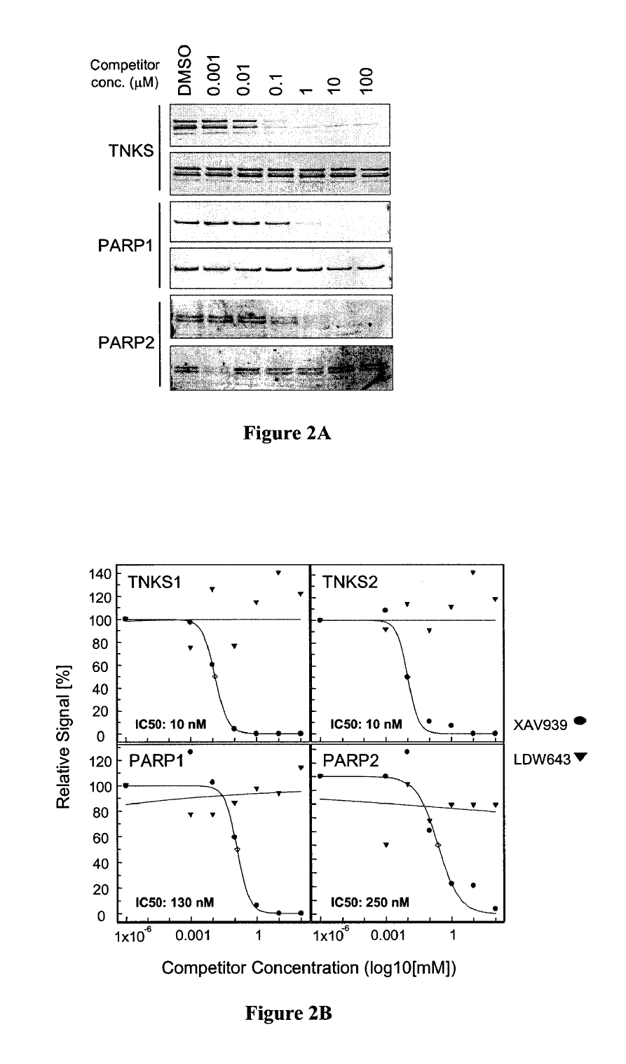

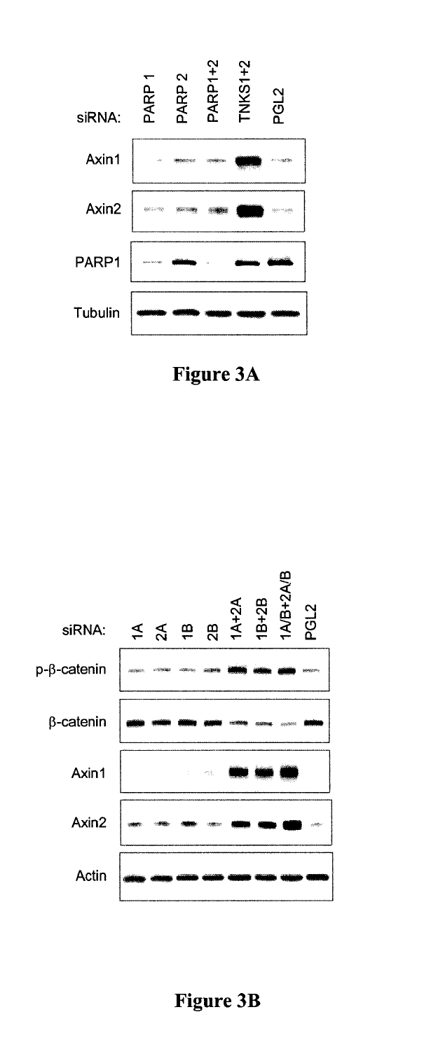

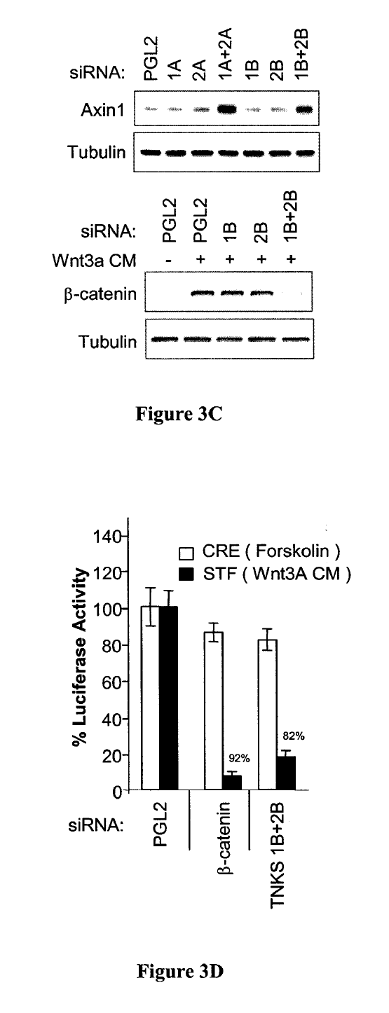

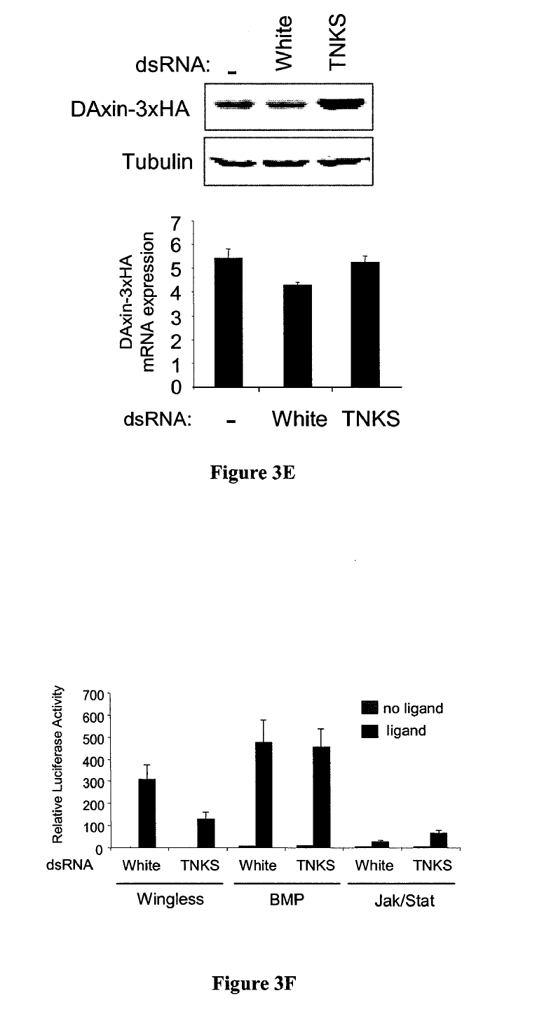

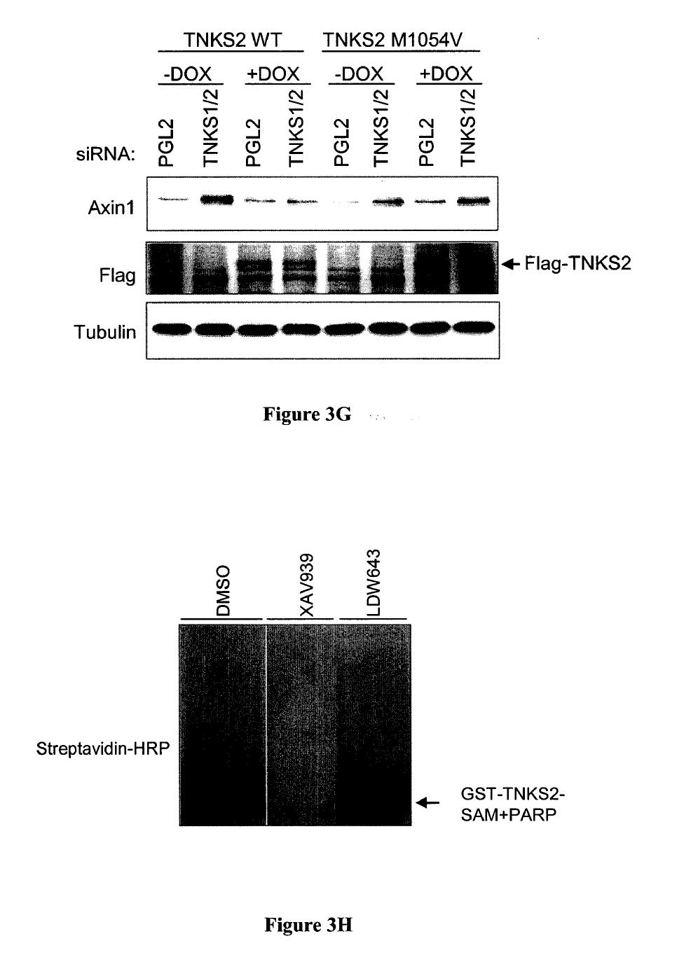

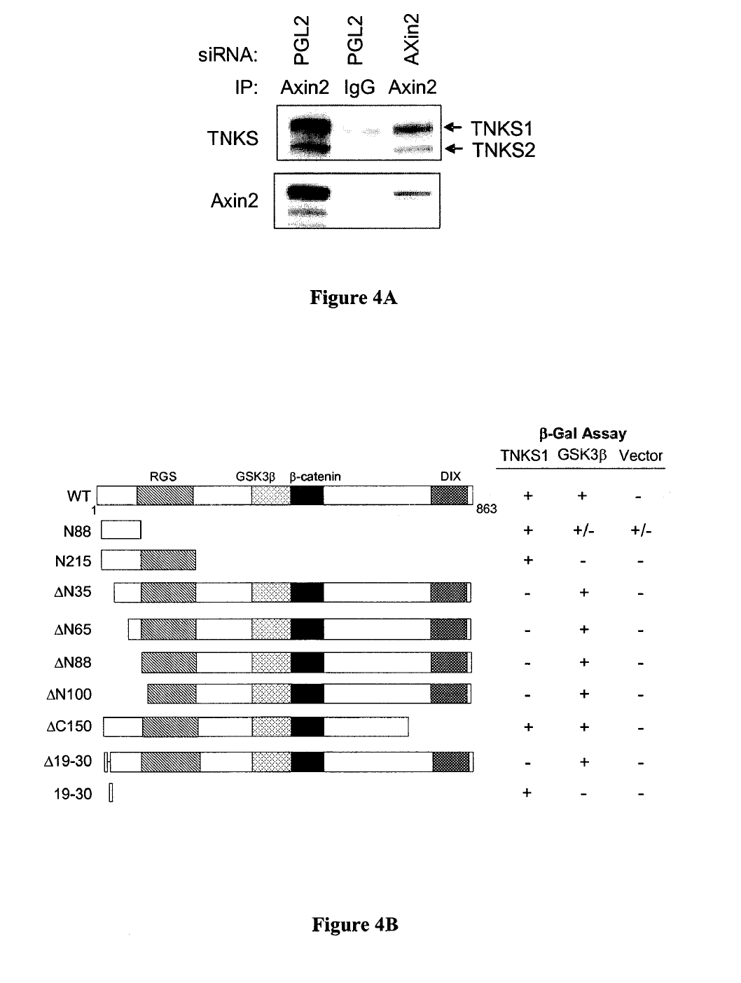

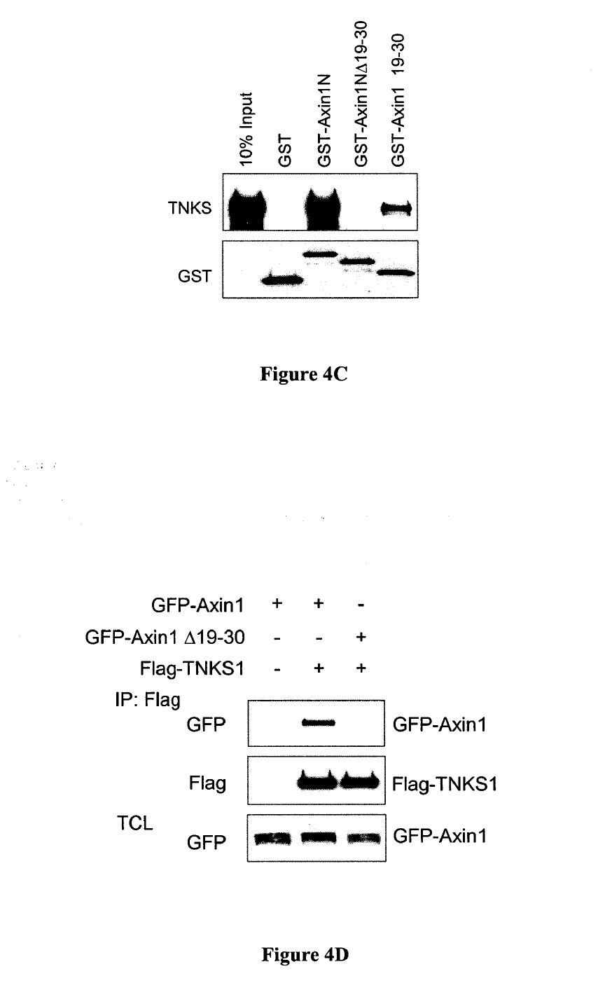

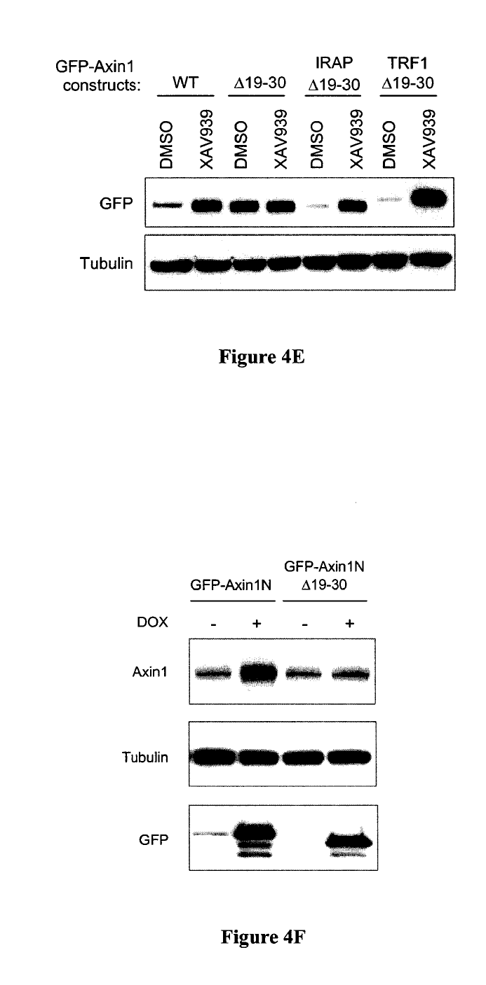

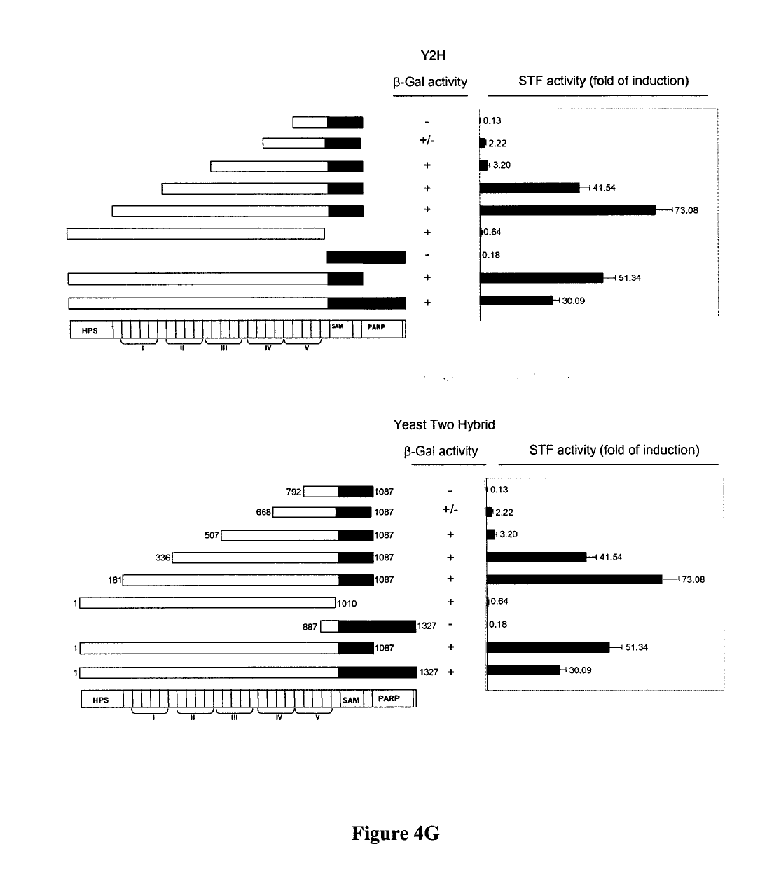

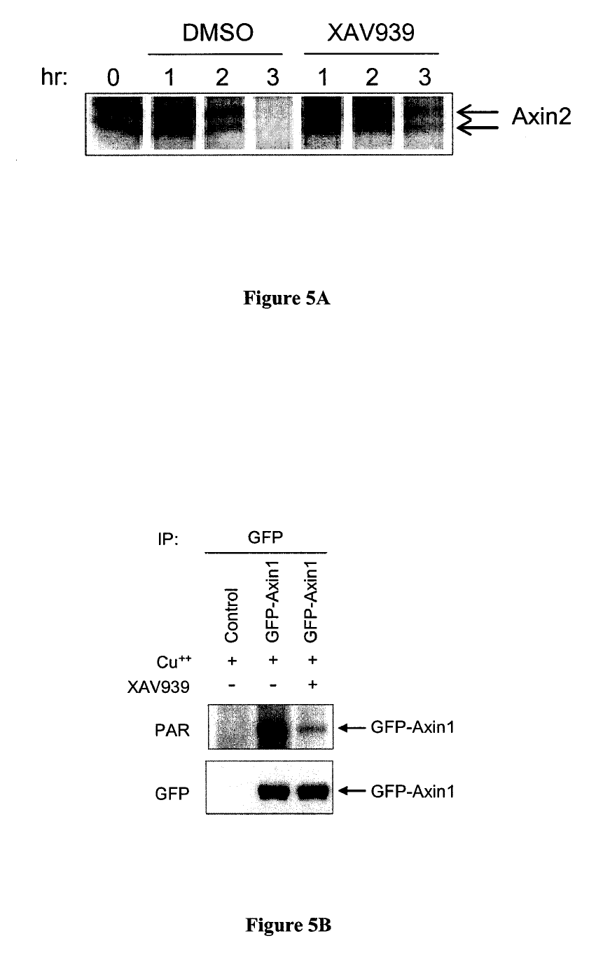

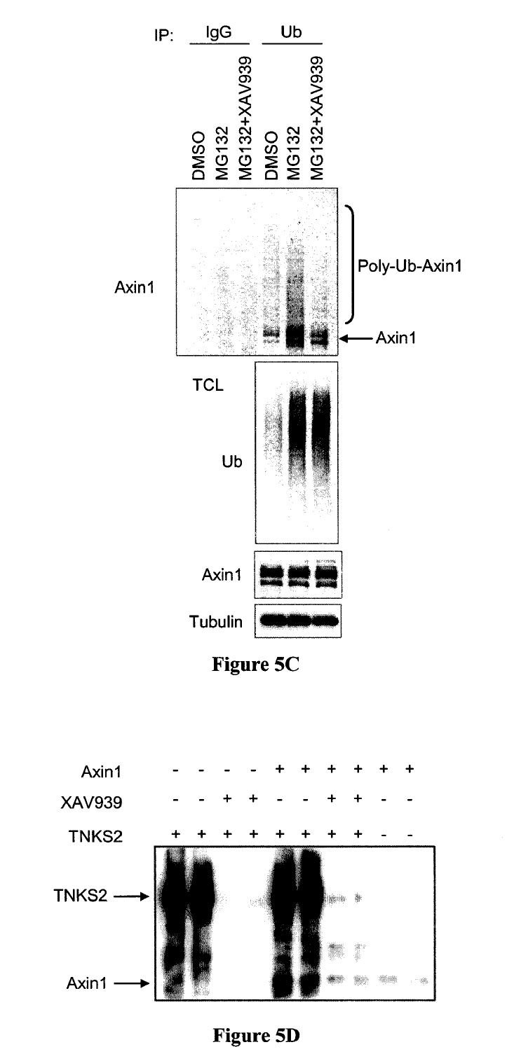

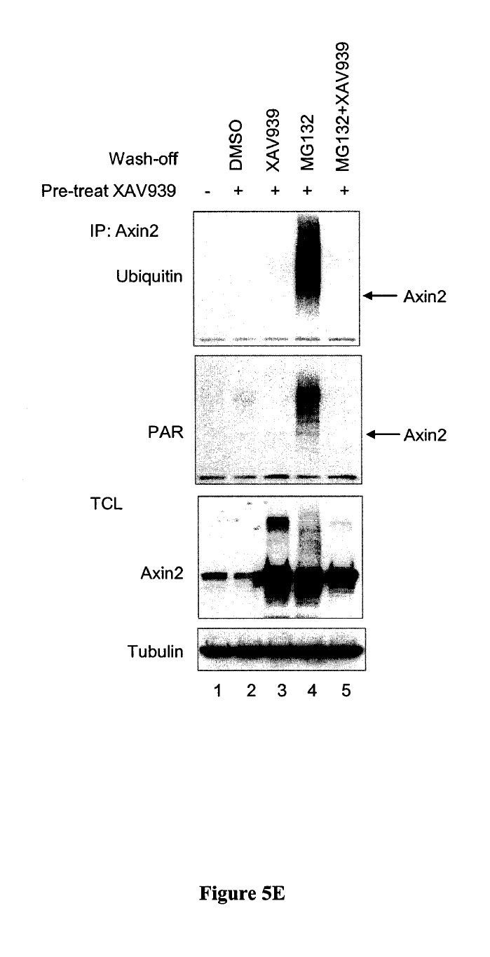

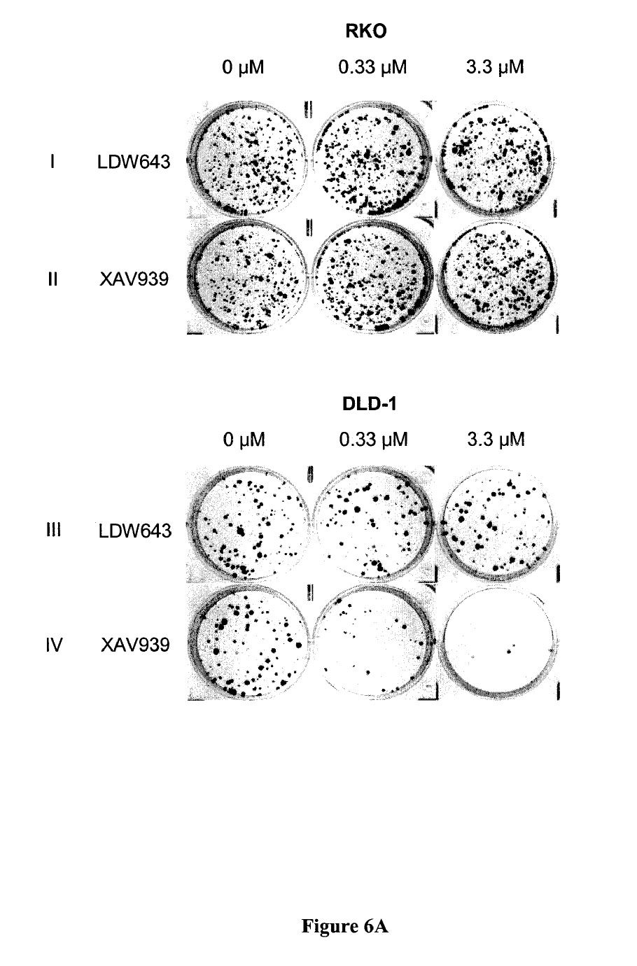

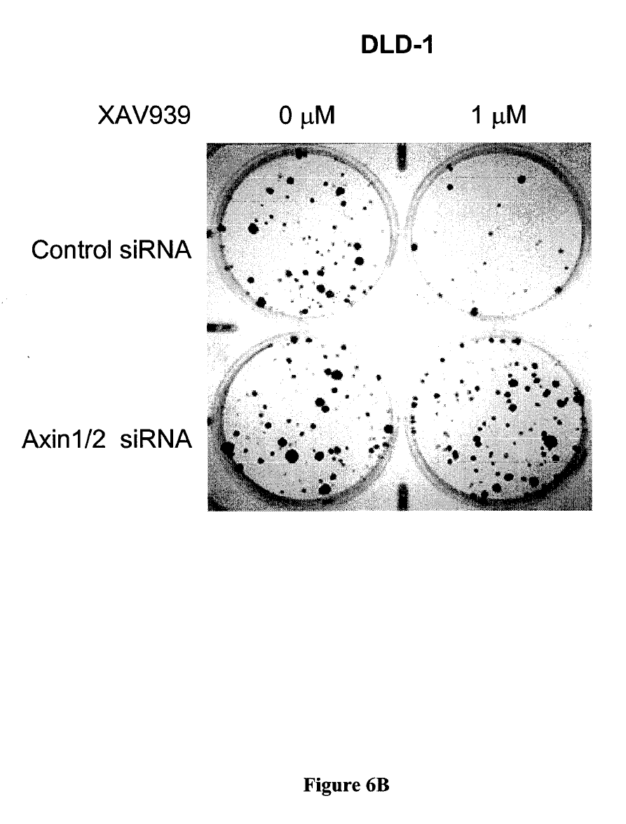

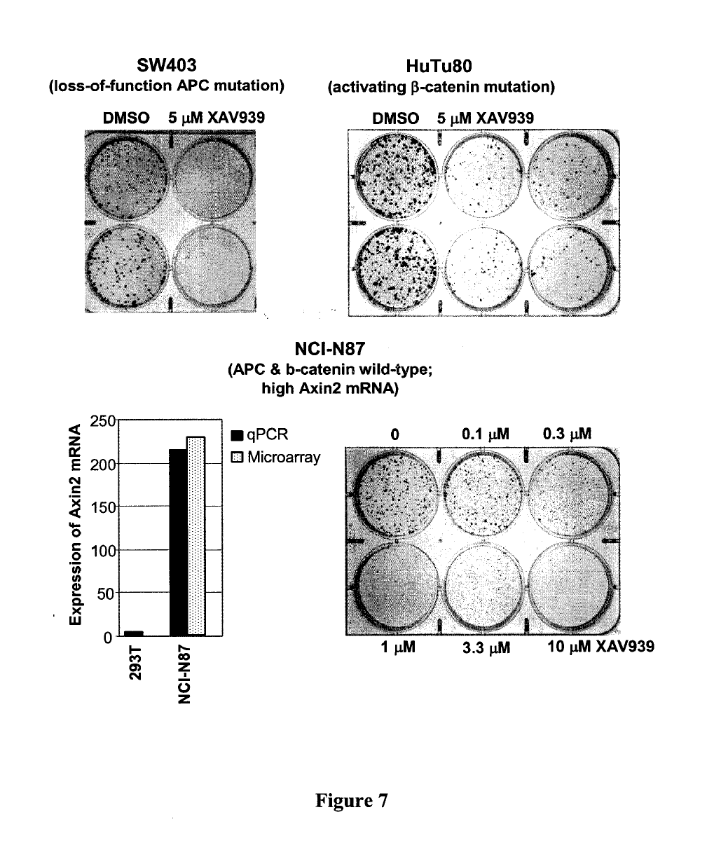

For clonogenic assay, cells were cultured in cell culture medium supplement with 10% fetal bovine serum, plated in 6-well plates at 1000-3000 cells/well density and treated with XAV939 for 12 days. Compound was replenished on every third day. Colonies were visualized by fixing and staining in crystal violet. For quantitative PCR, total RNA was isolated from cells to make cDNA using reverse transcription reaction. Quantitative PCR was performed using Axin2 specific probe-primers from Advanced Biosystems (ABI).

### DETAILED DESCRIPTION OF THE INVENTION

The present invention provides methods of diagnosing, ameliorating the symptoms of, protecting against, and treating Wnt signaling-related disorders (e.g., colorectal cancer), e.g., through use of modulators of Wnt pathway signal transduction, e.g., Axin stabilizers or destabilizers and/or TNKS modulators (e.g., small molecules (including, e.g., the compounds of the invention), inhibitory nucleic acids, fusion proteins, etc.).

Said methods may include administering to a subject in need thereof an effective amount of a modulator of Wnt pathway signal transduction, e.g., an Axin stabilizer and/or a TNKS modulator (e.g., small molecule (including, e.g., a compound of the invention), an inhibitory nucleic acid, fusion protein, etc., or any combination thereof), and a pharmaceutically acceptable carrier.

For purposes of the invention, and as explained in greater detail herein, “compounds of the invention” and like terminology, is used to describe compounds which inhibit, antagonize, mitigate, or weaken Wnt pathway signalling via Axin stabilization. The compounds include but are not limited to XAV939. The compounds can include other small molecule PARP inhibitors which preferentially inhibit the catalytic activity of TNKS1 and/or TNKS2 relative to that of other PARPs.

In one embodiment, an Axin stabilizer of the present invention (e.g., a compound of the invention (e.g., XAV939)) can be used to treat Wnt signaling disorders associated with aberrant upregulation of Wnt signaling (e.g., cancer, osteoarthritis, and polycystic kidney disease).

In another embodiment, Axin stabilization can be modulated such that Wnt signaling disorders associated with aberrant downregulation of Wnt signaling (e.g., osteoporosis, obesity, diabetes, and neuronal degenerative diseases) can be ameliorated. For example, the administration of a small molecule, fusion protein, or antibody capable of preventing the stabilization of Axin would in turn facilitate the stabilization of β-catenin (and therefore result in Wnt pathway signaling).

Said methods can be used at the cellular level, e.g., to treat epithelial cells having a Wnt receptor. For instance, the subject method can be used in treating or preventing basal cell carcinoma or other Wnt signaling-related disorders (e.g., those characterized by aberrant cell proliferation). The subject methods can also be used to prevent cellular proliferation, aberrant or otherwise, by inhibiting or agonizing the inhibition of Wnt signal transduction. Said methods can also be used for both human and animal subjects. Said methods can be used in vitro or in vivo.

The present invention also provides methods of modulating Wnt pathway signal transduction, e.g., through use of Axin stabilizers and/or TNKS modulators (e.g., small molecules, inhibitory nucleic acids, fusion proteins, etc.). In one embodiment, the methods of the present invention include inhibiting or agonizing the inhibition of Wnt pathway signal transduction, e.g., through use of Axin stabilizers and/or TNKS modulators (e.g., small molecules, inhibitory nucleic acids, fusion proteins, etc.). An agent which contributes to Axin stabilization (and thereby, to β-catenin phosphorylation and degradation) leads to the inhibition of Wnt pathway signal transduction; conversely, an agent which mitigates Axin stabilization (and thereby, stabilizes β-catenin) leads to an increase in Wnt pathway signal transduction.

Said methods of inhibiting or agonizing the inhibition of Wnt pathway signal transduction can be employed, e.g., in the regulation of repair and/or functional performance of a wide range of cells, tissues and organs, including normal cells, tissues, and organs. Non-limiting examples include regulation of neural tissues, bone and cartilage formation and repair, regulation of spermatogenesis, regulation of smooth muscle, regulation of lung, liver and other organs arising from the primitive gut, regulation of hematopoietic function, regulation of skin and hair growth, etc. The methods of the present invention can be performed in vitro or in vivo.

The present invention also provides methods of identifying and testing agonists and antagonists of Wnt pathway signal transduction, and methods of identifying and testing agonists and antagonists of Wnt pathway members (e.g., Axin, TNKS). The present discovery of inhibiting Wnt pathway signal transduction through the stabilization and raising of Axin protein levels, and of inhibiting Wnt pathway signal transduction through the inhibition of TNKS, is useful for identifying agents that will enhance or interfere with this stabilization, and thereby with Wnt pathway signaling, in vitro or in vivo. The present discovery is thereby also useful for discovering agents that can be used to treat disorders associated with the absence or presence of Axin stabilization (and the resulting modulation of Wnt pathway signal transduction).

In one embodiment, a method of identifying an agent capable of modulating Wnt pathway signal transduction comprises: a) contacting a biological sample in which the Wnt signaling pathway is active in the presence and absence of a test agent under conditions permitting Wnt signaling and in which Axin protein or stability levels can be measured; and b) measuring the levels of Axin protein or stability in both the presence and absence of said test agent, wherein (i) a decrease in Axin protein levels or stability in the presence of the test agent, relative to the absence of the test agent, identifies the test agent as an agonist of Wnt pathway signal transduction, and wherein (ii) an increase in Axin protein or stability levels in the presence of the test agent, relative to the absence of the test agent, identifies the test agent as an antagonist of Wnt pathway signal transduction.

In one embodiment, the agent can be a small molecule. In another embodiment, the agent can be an inhibitory nucleic acid. In another embodiment, the agent can be a fusion protein. In one embodiment, said small molecule, inhibitory nucleic acid, or fusion protein can act directly on Axin. In another embodiment, said small molecule, inhibitory nucleic acid, or fusion protein can act indirectly on Axin (e.g., can act on a binding partner of Axin, or Axin-associated protein, e.g., GSK3, β-catenin, APC, and Dishevelled, PP1, PP2A, Casein Kinase 1, LRP5/6).

The present invention includes a method for screening compounds useful for the treatment of Wnt signaling-related disorders (e.g., colorectal cancer), comprising contacting a cell exhibiting Wnt pathway signal transduction with a test agent and detecting a change in TNKS protein levels and/or in Axin protein levels and/or Axin stabilization.

In one embodiment, a method of identifying an agent useful for the treatment of Wnt signaling-related disorders, comprises: a) contacting a biological sample in which the Wnt signaling pathway is active in the presence and absence of a test agent under conditions permitting Wnt signaling and in which Axin protein levels can be measured; and b) measuring the levels of Axin protein in both the presence and absence of said test agent, wherein (i) a decrease in Axin protein levels in the presence of the test agent, relative to the absence of the test agent, identifies the test agent as useful for treating disorders associated with aberrant downregulation of Wnt signaling, and wherein (ii) an increase in Axin protein levels in the presence of the test agent, relative to the absence of the test agent, identifies the test agent as useful for treating disorders associated with aberrant upregulation of Wnt signaling.

In one embodiment, the agent can be a small molecule. In another embodiment, the agent can be an inhibitory nucleic acid. In another embodiment, the agent can be a fusion protein. In one embodiment, said small molecule, inhibitory nucleic acid, or fusion protein can act directly on Axin. In another embodiment, said small molecule, inhibitory nucleic acid, or fusion protein can act indirectly on Axin (e.g., can act on a binding partner of Axin, or Axin-associated protein, e.g., GSK3, β-catenin, APC, and Dishevelled, PP1, PP2A, Casein Kinase 1, LRP5/6).

In one embodiment, a method for identifying agents useful for the treatment of Wnt signaling-related disorders comprises contacting a cell in which the Wnt signaling pathway is active with a test agent and detecting a change in the Axin protein levels or stability.

The present invention includes pharmaceutical preparations comprising, as an active ingredient, a Wnt antagonist (e.g., an Axin stabilizer, a TNKS antagonist), such as described herein, formulated in an amount sufficient to (i) inhibit, in vivo, proliferation or other biological consequences of Wnt aberrant expression; and (ii) to diagnose, ameliorate the symptoms of, protect against, or treat Wnt signaling-related disorders. Said preparation can, e.g., include a compound, an inhibitory nucleic acid, or a fusion protein according to any embodiment of the present invention, or any combination thereof, in a pharmaceutically acceptable carrier.

In another embodiment, a method for inhibiting growth of a tumor cell is provided, which involves administering to a subject in need thereof an effective amount of a modulator of Wnt pathway signal transduction, e.g., an Axin stabilizer and/or a TNKS antagonist (e.g., small molecule (including, e.g., a compound of the invention), an inhibitory nucleic acid, fusion protein, etc., or any combination thereof), and a pharmaceutically acceptable carrier. By way of non-limiting example, and as explained further herein, the compounds of the invention (e.g., Compound I) are capable of inhibiting Wnt signaling in both colon cancer cells with APC deficiencies, and cells lines with an intact Wnt signaling pathway. Also by way of non-limiting example, and as explained further herein, the compounds of the invention (e.g., Compound I) are capable of inhibiting the growth of colon cancer cells in in vitro cell culture assays.

Further provided is a method for inducing apoptosis in a tumor cell, which includes administering to a subject in need thereof an effective amount of a modulator of Wnt pathway signal transduction, e.g., an Axin stabilizer and/or a TNKS antagonist (e.g., small molecule (including, e.g., a compound of the invention), an inhibitory nucleic acid, fusion protein, etc., or any combination thereof), and a pharmaceutically acceptable carrier.

In one embodiment of the methods of the invention, an Axin stabilizer is administered, such as a compound of the invention. In said embodiment, the Axin stabilizer leads to an increase in Axin protein levels in the cell or system to which it is administered. As a result, β-catenin undergoes a concomitant phosphorylation and degradation via a GSK3 mechanism. The combination of Axin stabilization and β-catenin degradation results in inhibition of Wnt pathway signaling. At least one utility of said embodiment is the treatment of disorders in which Wnt signaling levels are aberrantly high (e.g., colon cancer). Another utility is the inhibition of growth of a tumor cell, and/or induction of apoptosis in a tumor cell.

In one embodiment of the methods of the invention, an Axin stabilizer is administered which acts by increasing Axin phosphorylation by GSK3. In one embodiment, XAV939 is capable of inducing increasing Axin phosphorylation by GSK3, thereby stabilizing Axin and increasing Axin protein levels, and thereby inhibiting Wnt signal transduction.

The present invention includes methods for identifying or predicting the predisposition or likelihood of subjects afflicted with Wnt signaling-related disorders (e.g., colorectal cancer) to benefit from a treatment regiment that includes “TNKS inhibitor,” “Axin stabilizers,” or the like (including, e.g., the compounds of the invention, or fusion proteins or inhibitory nucleotides capable of inhibiting the catalytic activity of TNKS).

As explained further herein, said methods involve first diagnosing a subject afflicted with a Wnt signaling-related disorder (e.g., colorectal cancer), then detecting whether or not said subject demonstrates the presence of one or more biomarkers of a disorder associated with aberrant Axin stabilization and/or β-catenin degradation. Presence of said biomarker indicates that said subject would benefit from a treatment regiment that includes “TNKS inhibitor,” “Axin stabilizers,” or the like. Non-limiting examples of said biomarkers include (i) truncating mutations of the tumor suppressor APC; (ii) Axin1 and Axin2 mutations; (iii) β-catenin overexpression; and increased formation of the Axin-GSK3 complex.

### DEFINITIONS

The term “treat,” “treated,” “treating” or “treatment” includes the diminishment or alleviation of at least one symptom associated or caused by the state, disorder or disease being treated. In certain embodiments, the treatment comprises the induction of a Wnt signaling-related disorder, followed by the activation of the compound of the invention, which would in turn diminish or alleviate at least one symptom associated or caused by the Wnt signaling-related disorder being treated. For example, treatment can be diminishment of one or several symptoms of a disorder or complete eradication of a disorder.

The term “use” includes any one or more of the following embodiments of the invention, respectively: the use in the treatment of Wnt signaling-related disorders; the use for the manufacture of pharmaceutical compositions for use in the treatment of these diseases, e.g., in the manufacture of a medicament; methods of use of compounds of the invention in the treatment of these diseases; pharmaceutical preparations having compounds of the invention for the treatment of these diseases; and compounds of the invention for use in the treatment of these diseases; as appropriate and expedient, if not stated otherwise. In particular, diseases to be treated and are thus preferred for use of a compound of the present invention are selected from cancer (e.g., colon cancer) and other proliferative diseases, osteoporosis, and schizophrenia, as well as those diseases that depend on the activity of Wnt signaling.

The term “Wnt signaling-related disorders” means diseases and conditions associated with aberrant Wnt signaling, including but not limited to cancers (e.g., colorectal carcinomas (CRCs), melanoma, breast, liver, lung, and gastric cancer; other, non-oncogenic proliferative diseases, such as proliferative skin disorders (e.g., psoriasis, dermatitis); osteoporosis; osteoarthritis; fibrosis; schizophrenia; vascular disease; cardiac disease; and neurodegenerative diseases such as Alzheimer's disease. Aberrant upregulation of Wnt signaling is associated with cancer, osteoarthritis, and polycystic kidney disease, while aberrant downregulation of Wnt signaling has been linked to osteoporosis, obesity, diabetes, and neuronal degenerative diseases.

As used herein, “Wnt signaling-related cancers” include but are not limited to colorectal carcinomas (CRCs), melanoma, breast, liver, lung, and gastric cancer. The term “Wnt-related cancer,” as used herein includes malignant medulloblastoma and other primary CNS malignant neuroectodermal tumors, rhabdomyosarcoma, lung cancer, and in particular small cell lung cancer, gut-derived tumors, including but not limited to cancer of the esophagus, stomach, pancreas, and biliary duct system; prostate and bladder cancers, colon cancer, and liver cancer.

The term “Wnt antagonist” as used herein includes inhibitors, or agonizers of inhibition, of Wnt signal transduction, as described herein. In one or more embodiment, said Wnt antagonists act via Axin stabilization. In one or more embodiment, said Wnt antagonists act via TNKS antagonism (e.g., by inhibiting the catalytic ability of TNKS, and thereby stabilizing Axin). Wnt antagonists include but are not limited to small molecules (including, e.g., the compounds of the invention), inhibitory nucleic acids, and fusion proteins.

The term “TNKS antagonist,” “TNKS inhibitor,” or the like, as used herein means an agent capable of increasing the stability of Axin. “TNKS antagonist,” “TNKS inhibitor,” and the like can include Axin stabilizers such as the compounds of the invention. TNKS antagonists preferably act by reducing or inhibiting the catalytic activity of TNKS proteins (e.g., their ability to PARsylate target proteins such as Axin, as well as their ability to autoparsylate), and not by reducing TNKS protein or transcript levels. TNKS antagonists are also thought to inhibit Wnt signaling, for reasons which include that Axin is a negative regulator of Wnt signalling, and TNKS interacts with Axin (e.g., knocking down TNKS stabilizes and increases Axin protein levels). TNKS antagonists increase phospho-β-catenin, decrease cytosolic β-catenin, and impact β-catenin target genes in a fashion analogous to β-catenin siRNA.

The term “Axin stabilizer” as used herein means an agent capable of increasing the stability of Axin. This leads to accelerated phosphorylation, and degradation, of β-catenin. Axin-stabilizers are also thought to inhibit Wnt signaling, for reasons which include that Axin is a negative regulator of Wnt signaling. Axin stabilizers (e.g., the compounds of the invention (e.g., XAV939)) contribute to a decrease in total β-catenin, but an increase in phosphor-β-catenin, in a cell. For purposes of the present invention, the term “Axin” is used interchangeably for Axin1 and Axin2, and the Axin stabilizers of the invention are capable of stabilizing and increasing the protein levels of both Axin1 and Axin2. Furthermore, “Axin,” as used herein, can apply to Axin1 and/or Axin2 from human, mouse, rat, or other species.

The term “Axin-associated protein” as used herein means a protein member of the Wnt signalling pathway with which Axin associates (e.g., binds directly or indirectly, is a target of, forms a protein complex with, and/or exerts an influence on) under normal conditions. Said Axin-associated proteins include but are not limited to GSK3, β-catenin, APC, and Dishevelled, PP1, PP2A, Casein Kinase 1, LRP5/6.

The term “compounds of the invention” and like terminology, as defined further herein, are used herein to describe compounds which can be used, for instance, to stabilize Axin (and to thereby inhibit Wnt pathway signaling). The compounds include but are not limited to XAV939.

The compounds can include other small molecule PARP inhibitors which preferentially inhibit the catalytic activity of TNKS1 and/or TNKS2 relative to that of other PARPs.

“Cure” as used herein means to lead to the remission of the disorder, e.g., a Wnt signaling-related disorder, e.g., osteoporosis, schizophrenia, vascular disease, cardiac disease, or a neurodegenerative disease, through treatment.

The terms “prophylaxis” or “prevention” means impeding the onset or recurrence of a disorder, e.g., a Wnt signaling-related disorder.

As used herein, the term “medical condition” includes, but is not limited to, any condition or disease manifested as one or more physical and/or psychological symptoms for which treatment is desirable, and includes previously and newly identified diseases and other disorders.

As used herein, the administration of an agent or drug to a subject or patient includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

As used herein, “modulate” indicates the ability to control or influence directly or indirectly, and by way of non-limiting examples, can alternatively mean inhibit or stimulate, agonize or antagonize, hinder or promote, and strengthen or weaken.

As used herein a “small organic molecule,” or “small molecule,” is an organic compound (or organic compound complexed with an inorganic compound (e.g., metal) that has a molecular weight of less than 3 kilodaltons, and preferably less than 1.5 kilodaltons.

As used herein, the term “effective amount” of a compound is a quantity sufficient to achieve a desired therapeutic and/or prophylactic effect, for example, an amount which results in the prevention of or a decrease in the symptoms associated with a disease that is being treated, e.g., disorders associated with aberrant Wnt signaling. The amount of compound administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. Typically, an effective amount of the compounds of the present invention, sufficient for achieving a therapeutic or prophylactic effect, range from about 0.000001 mg per kilogram body weight per day to about 10,000 mg per kilogram body weight per day. Preferably, the dosage ranges are from about 0.0001 mg per kilogram body weight per day to about 100 mg per kilogram body weight per day. The compounds of the present invention can also be administered in combination with each other, or with one or more additional therapeutic compounds.

The term “subject” is intended to include organisms, e.g., prokaryotes and eukaryotes, which are capable of suffering from or afflicted with a disease, disorder or condition associated with aberrant Wnt signaling. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from cancer (e.g., colon cancer) and other proliferative diseases, osteoporosis, and schizophrenia, and other diseases or conditions described herein (e.g., a Wnt signaling-related disorder). In another embodiment, the subject is a cell.

As used herein, the term “aryl” is defined as an aromatic radical having 6 to 14 ring carbon atoms, and no ring heteroatoms. The aryl group may be monocyclic or fused bicyclic or tricyclic. It may be unsubstituted or substituted by one or more, preferably one or two, substituents, wherein the substituents are as described herein. As defined herein, the aryl moiety may be completely aromatic regardless of whether it is monocyclic or bicyclic. However, if it contains more than one ring, as defined herein, the term aryl includes moieties wherein at least one ring is completely aromatic while the other ring(s) may be partially unsaturated or saturated or completely aromatic.

“Het” as used herein, refers to heteroaryl and heterocyclic compounds containing at least one S, O or N ring heteroatom. More specifically, “Het” is a 5-7 membered heterocyclic ring containing 1-4 heteroatoms selected from N, O and S, or an 8-12 membered fused ring system including at least one 5-7 membered heterocyclic ring containing 1, 2 or 3 heteroatoms selected from N, O, and S. Examples of het, as used herein, include but are not limited to unsubstituted and substituted pyrrolidyl, tetrahydrofuryl, tetrahydrothiofuryl, piperidyl, piperazyl, purinyl, tetrahydropyranyl, morpholino, 1,3-diazapanyl, 1,4-diazapanyl, 1,4-oxazepanyl, 1,4-oxathiapanyl, furyl, thienyl, pyrryl, pyrrolyl, pyrazolyl, triazolyl, tetrazolyl, indazolyl, oxadiazolyl, imidazolyl, pyrrolidyl, pyrrolidinyl, thiazolyl, oxazolyl, pyridyl, pyrazolyl, pyrazinyl, pyrimidinyl, isoxazolyl, pyrazinyl, quinolyl, isoquinolyl, pyridopyrazinyl, pyrrolopyridyl, furopyridyl, indolyl, benzofuryl, benzothiofuryl, benzoindolyl, benzothienyl, pyrazolyl, piperidyl, piperazinyl, indolinyl, morpholinyl, benzoxazolyl, pyrroloquinolyl, pyrrolo[2,3-b]pyridinyl, benzotriazolyl, oxobenzo-oxazolyl, benco[1,3]dioxolyl, benxzoimidazolyl, quinolinyl, indanyl and the like. Heteroaryls are within the scope of the definition of het. Examples of heteroaryls are pyridyl, pyrimidinyl, quinolyl, thiazolyl and benzothiazolyl. The most preferred het are pyridyl, pyrimidinyl and thiazolyl. The het may be unsubstituted or substituted as described herein. It is preferred that it is unsubstituted or if substituted it is substituted on a carbon atom by halogen, especially fluorine or chlorine, hydroxy, C1-C4 alkyl, such as methyl and ethyl, C1-C4 alkoxy, especially methoxy and ethoxy, nitro, —O—C(O)—C1-C4alkyl or —C(O)—O—C1-C4alkyl, SCN or nitro or on a nitrogen atom by C1-C4 alkyl, especially methyl or ethyl, —O—C(O)—C1-C4alkyl or —C(O)—O—C1-C4alkyl, such as carbomethoxy or carboethoxy.

When two substituents together with a commonly bound nitrogen are het, it is understood that the resulting heterocyclic ring is a nitrogen-containing ring, such as aziridine, azetidine, azole, piperidine, piperazine, morphiline, pyrrole, pyrazole, thiazole, oxazole, pyridine, pyrimidine, isoxazole, and the like, wherein such het may be unsubstituted or substituted as defined hereinabove.

Halo is halogen, and may be fluorine, chlorine, bromine or iodine, especially fluorine and chlorine.

Unless otherwise specified, the term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl(alicyclic) groups (cyclopropyl, cyclopentyl, cyclohexyl, cycloheptyl, cyclooctyl), alkyl substituted cycloalkyl groups, and cycloalkyl substituted alkyl groups. The term “alkyl” also includes alkenyl groups and alkynyl groups. Furthermore, the expression “Cx-Cy-alkyl”, wherein x is 1-5 and y is 2-10 indicates a particular alkyl group (straight- or branched-chain) of a particular range of carbons. For example, the expression C1-C4-alkyl includes, but is not limited to, methyl, ethyl, propyl, butyl, isopropyl, tert-butyl, and isobutyl and sec-butyl. Moreover, the term C3-7-cycloalkyl includes, but is not limited to, cyclopropyl, cyclopentyl, cyclohexyl and cycloheptyl. As discussed below, these alkyl groups, as well as cycloalkyl groups, may be further substituted.

The term alkyl further includes alkyl groups which can further include oxygen, nitrogen, sulfur or phosphorous atoms replacing one or more carbons of the hydrocarbon backbone. In an embodiment, a straight chain or branched chain alkyl has 10 or fewer carbon atoms in its backbone (e.g., C1-C10 for straight chain, C3-C10 for branched chain), and more preferably 6 or fewer carbons. Likewise, preferred cycloalkyls have from 4-7 carbon atoms in their ring structure, and more preferably have 5 or 6 carbons in the ring structure.

Moreover, alkyl (e.g., methyl, ethyl, propyl, butyl, pentyl, hexyl, etc.) includes both “unsubstituted alkyl” and “substituted alkyl”, the latter of which refers to alkyl moieties having substituents replacing a hydrogen on one or more carbons of the hydrocarbon backbone, which allow the molecule to perform its intended function.

A “cycloalkyl” group means C3 to C10 cycloalkyl having 3 to 10 ring carbon atoms and may be, for example, cyclopropyl, cyclobutyl, cyclopentyl, cyclohexyl, cycloheptyl or cyclooctyl, cyclononyl and the like. The cycloalkyl group may be monocyclic or fused bicyclic. Moreover, the preferred cycloalkyl group is cyclopentyl or cyclohexyl. Most preferably, cycloalkyl is cyclohexyl. The cycloalkyl group may be fully saturated or partially unsaturated, although it is preferred that it is fully saturated. As defined herein, it excludes aryl groups. The cycloalkyl groups may be unsubstituted or substituted with any of the substituents defined below, preferably halo, hydroxy or C1-C6 alkyl such as methyl.

The term “substituted” is intended to describe moieties having substituents replacing a hydrogen on one or more atoms, e.g. C, O or N, of a molecule. Such substitutents can include electron-withdrawing groups or electron-withdrawing atoms. Such substituents can include, for example, oxo, alkyl, alkoxy, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclic, alkylaryl, morpholino, phenol, benzyl, phenyl, piperizine, cyclopentane, cyclohexane, pyridine, 5H-tetrazole, triazole, piperidine, or an aromatic or heteroaromatic moiety, and any combination thereof.

Unsubstituted is intended to mean that hydrogen is the only substituent.

Except as described herein, any of the above defined aryl, het, alkyl, alkenyl, alkynyl, or cycloalkyl, may be unsubstituted or independently substituted by up to four, preferably one, two or three substituents, selected from the group consisting of: halo (such as Cl or Br); hydroxy; lower alkyl (such as C1-C3 alkyl); lower alkyl which may be substituted with any of the substituents defined herein; lower alkenyl; lower alkynyl; lower alkanoyl; lower alkoxy (such as methoxy); aryl (such as phenyl or naphthyl); substituted aryl (such as fluoro phenyl or methoxy phenyl); aryl lower alkyl such as benzyl, amino, mono or di-lower alkyl (such as dimethylamino); lower alkanoyl amino acetylamino; amino lower alkoxy (such as ethoxyamine); nitro; cyano; cyano lower alkyl; carboxy; lower carbalkoxy (such as methoxy carbonyl; n-propoxy carbonyl or iso-propoxy carbonyl), lower aryloyl, such as benzoyl; carbamoyl; N-mono- or N,N di-lower alkyl carbamoyl; lower alkyl carbamic acid ester; amidino; guanidine; ureido; mercapto; sulfo; lower alkylthio; sulfoamino; sulfonamide; benzosulfonamide; sulfonate; sulfanyl lower alkyl (such as methyl sulfanyl); sulfoamino; aryl sulfonamide; halogen substituted or unsubstituted aryl sulfonate (such as chloro-phenyl sulfonate); lower alkylsulfinyl; arylsulfinyl; aryl-lower alkylsulfinyl; lower alkylarylsulfinyl; lower alkanesulfonyl; arylsulfonyl; aryl-lower alkylsulfonyl; lower aryl alkyl; lower alkylarylsulfonyl; halogen-lower alkylmercapto; halogen-lower alkylsulfonyl; such as trifluoromethane sulfonyl; phosphono(—P(═O)(OH)2); hydroxy-lower alkoxy phosphoryl or di-lower alkoxyphosphoryl; urea and substituted urea; alkyl carbamic acid ester or carbamates (such as ethyl-N-phenyl-carbamate); or lower alkyl (e.g. methyl, ethyl or propyl).

In an embodiment, the above mentioned alkyl, cycloalkyl, and aryl groups are independently unsubstituted or are substituted by lower alkyl, aryl, aryl lower alkyl, carboxy, lower carbalkoxy and especially halogen, —OH, —SH, —OCH3, —SCH3, —CN, —SCN or nitro.

As defined herein the term “lower alkyl”, when used alone or in combination refers to alkyl containing 1-6 carbon atoms. The alkyl group may be branched or straight-chained, and is as defined hereinabove.

The term “alkenyl” indicates a hydrocarbyl group containing at least one carbon-carbon double bond, and includes unsaturated aliphatic groups analogous in length and possible substitution to the alkyls described above. As defined herein, it may be unsubstituted or substituted with the substituents described herein. The carbon-carbon double bonds may be between any two carbon atoms of the alkenyl group. It is preferred that it contains 1 or 2 carbon-carbon double bonds and more preferably one carbon-carbon double bond. The alkenyl group may be straight chained or branched. Examples include but are not limited to ethenyl, 1-propenyl, 2-propenyl, 1-butenyl, 2-butenyl, 2-methyl-1-propenyl, 1,3-butadienyl, and the like. The term “lower alkenyl” refers to a alkenyl group which contains 2-6 carbon atoms.

For example, the term “alkenyl” includes straight-chain alkenyl groups (e.g., ethenyl, propenyl, butenyl, pentenyl, hexenyl, heptenyl, octenyl, nonenyl, decenyl, etc.), branched-chain alkenyl groups, cycloalkenyl(alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups that include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C2-C6 for straight chain, C3-C6 for branched chain). Likewise, cycloalkenyl groups may have from 3-8 carbon atoms in their ring structure, and more preferably have 5 or 6 carbons in the ring structure. The term C2-C6 includes alkenyl groups containing 2 to 6 carbon atoms.

Moreover, the term alkenyl includes both “unsubstituted alkenyls” and “substituted alkenyls”, the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “aryl alkyl” refers to a aryl group connected to the main chain by a bridging alkylene group. Examples include but are not limited to benzyl, phenethyl, naphthylmethyl, and the like. Similarly, cyano alkyl group refers to a cyano group connected to the main chain by a bridging alkylene group.

The term “alkyl aryl” on the other hand, refers to an alkyl group bridged to the main chain through a phenylene group. Examples include but are not limited to methylphenyl, ethylphenyl, and the like.

As used herein, the term “alkanoyl” refers to an alkyl chain in which one of the carbon atoms is replaced by a C═O group. The C═O group may be present at one of the ends of the substituent or in the middle of the moiety. Examples include but are not limited to formyl, acetyl, 2-propanoyl, 1-propanoyl and the like.

The term “lower thioalkyl” refers to an alkyl group, as defined herein, connected to the main chain by a sulfur atom. Examples include but are not limited to thiomethyl (or mercapto methyl), thioethyl(mercapto ethyl) and the like.

The term “lower carbalkoxy” or synonym thereto refers to an alkoxycarbonyl group, where the attachment to the main chain is through the aryl group (C(O)). Examples include but are not limited to methoxy carbonyl, ethoxy carbonyl, and the like.

It is to be understood that the terminology C(O) refers to a —C═O group, whether it be ketone, aldehydre or acid or acid derivative. Similarly, S(O) refers to a —S═O group.

The term “alkynyl” includes unsaturated aliphatic groups analogous in length and possible substitution to the alkyls described above, but which contain at least one triple bond.

For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups that include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C2-C6 for straight chain, C3-C6 for branched chain). The term C2-C6 includes alkynyl groups containing 2 to 6 carbon atoms.

Moreover, the term alkynyl includes both “unsubstituted alkynyls” and “substituted alkynyls”, the latter of which refers to alkynyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “amine” or “amino” should be understood as being broadly applied to both a molecule, or a moiety or functional group, as generally understood in the art, and can be primary, secondary, or tertiary. The term “amine” or “amino” includes compounds where a nitrogen atom is covalently bonded to at least one carbon, hydrogen or heteroatom. The terms include, for example, but are not limited to, “alkyl amino,” “arylamino,” “diarylamino,” “alkylarylamino,” “alkylaminoaryl,” “arylaminoalkyl,” “alkaminoalkyl,” “amide,” “amido,” and “aminocarbonyl.” The term “alkyl amino” comprises groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group which is bound to at least one alkyl group and at least one aryl group. The term “alkaminoalkyl” refers to an alkyl, alkenyl, or alkynyl group bound to a nitrogen atom which is also bound to an alkyl group.

The term “amide,” “amido” or “aminocarbonyl” includes compounds or moieties which contain a nitrogen atom which is bound to the carbon of a carbonyl or a thiocarbonyl group. The term includes “alkaminocarbonyl” or “alkylaminocarbonyl” groups which include alkyl, alkenyl, aryl or alkynyl groups bound to an amino group bound to a carbonyl group. It includes arylaminocarbonyl and arylcarbonylamino groups which include aryl or heteroaryl moieties bound to an amino group which is bound to the carbon of a carbonyl or thiocarbonyl group. The terms “alkylaminocarbonyl,” “alkenylaminocarbonyl,” “alkynylaminocarbonyl,” “arylaminocarbonyl,” “alkylcarbonylamino,” “alkenylcarbonylamino,” “alkynylcarbonylamino,” and “arylcarbonylamino” are included in term “amide.” Amides also include urea groups (aminocarbonylamino) and carbamates (oxycarbonylamino).

The term “aryl” includes groups, including 5- and 6-membered single-ring aromatic groups that can include from zero to four heteroatoms, for example, phenyl, pyrrole, furan, thiophene, thiazole, isothiaozole, imidazole, triazole, tetrazole, pyrazole, oxazole, isoxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, anthryl, phenanthryl, napthridine, indole, benzofuran, purine, benzofuran, deazapurine, or indolizine.

Those aryl groups having heteroatoms in the ring structure can also be referred tows “aryl heterocycles”, “heterocycles,” “heteroaryls” or “heteroaromatics.” The aromatic ring can be substituted at one or more ring positions with such substituents as described above, as for example, alkyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminoacarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “heteroaryl,” as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

The term “heterocycle” or “heterocyclyl” as used herein is intended to mean a 5- to 10-membered aromatic or nonaromatic heterocycle containing from 1 to 4 heteroatoms selected from the group consisting of O, N and S, and includes bicyclic groups. “Heterocyclyl” therefore includes the above mentioned heteroaryls, as well as dihydro and tetrathydro analogs thereof. Further examples of “heterocyclyl” include, but are not limited to the following: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom.

The term “acyl” includes compounds and moieties which contain the acyl radical (CH3CO—) or a carbonyl group. The term “substituted acyl” includes acyl groups where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “acylamino” includes moieties wherein an acyl moiety is bonded to an amino group. For example, the term includes alkylcarbonylamino, arylcarbonylamino, carbamoyl and ureido groups.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups and may include cyclic groups such as cyclopentoxy. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “carbonyl” or “carboxy” includes compounds and moieties which contain a carbon connected with a double bond to an oxygen atom, and tautomeric forms thereof. Examples of moieties that contain a carbonyl include aldehydes, ketones, carboxylic acids, amides, esters, anhydrides, etc. The term “carboxy moiety” or “carbonyl moiety” refers to groups such as “alkylcarbonyl” groups wherein an alkyl group is covalently bound to a carbonyl group, “alkenylcarbonyl” groups wherein an alkenyl group is covalently bound to a carbonyl group, “alkynylcarbonyl” groups wherein an alkynyl group is covalently bound to a carbonyl group, “arylcarbonyl” groups wherein an aryl group is covalently attached to the carbonyl group. Furthermore, the term also refers to groups wherein one or more heteroatoms are covalently bonded to the carbonyl moiety. For example, the term includes moieties such as, for example, aminocarbonyl moieties, (wherein a nitrogen atom is bound to the carbon of the carbonyl group, e.g., an amide), aminocarbonyloxy moieties, wherein an oxygen and a nitrogen atom are both bond to the carbon of the carbonyl group (e.g., also referred to as a “carbamate”). Furthermore, aminocarbonylamino groups (e.g., ureas) are also include as well as other combinations of carbonyl groups bound to heteroatoms (e.g., nitrogen, oxygen, sulfur, etc. as well as carbon atoms). Furthermore, the heteroatom can be further substituted with one or more alkyl, alkenyl, alkynyl, aryl, aralkyl, acyl, etc. moieties.

The term “thiocarbonyl” or “thiocarboxy” includes compounds and moieties which contain a carbon connected with a double bond to a sulfur atom. The term “thiocarbonyl moiety” includes moieties that are analogous to carbonyl moieties. For example, “thiocarbonyl” moieties include aminothiocarbonyl, wherein an amino group is bound to the carbon atom of the thiocarbonyl group, furthermore other thiocarbonyl moieties include, oxythiocarbonyls (oxygen bound to the carbon atom), aminothiocarbonylamino groups, etc.

The term “ether” includes compounds or moieties that contain an oxygen bonded to two different carbon atoms or heteroatoms. For example, the term includes “alkoxyalkyl” which refers to an alkyl, alkenyl, or alkynyl group covalently bonded to an oxygen atom that is covalently bonded to another alkyl group.

The term “ester” includes compounds and moieties that contain a carbon or a heteroatom bound to an oxygen atom that is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above.

The term “thioether” includes compounds and moieties which contain a sulfur atom bonded to two different carbon or hetero atoms. Examples of thioethers include, but are not limited to alkthioalkyls, alkthioalkenyls, and alkthioalkynyls. The term “alkthioalkyls” include compounds with an alkyl, alkenyl, or alkynyl group bonded to a sulfur atom that is bonded to an alkyl group. Similarly, the term “alkthioalkenyls” and alkthioalkynyls” refer to compounds or moieties wherein an alkyl, alkenyl, or alkynyl group is bonded to a sulfur atom which is covalently bonded to an alkynyl group.

The term “hydroxy” or “hydroxyl” includes groups with an —OH or —O—.

The term “halogen” includes fluorine, bromine, chlorine, iodine, etc. The term “perhalogenated” generally refers to a moiety wherein all hydrogens are replaced by halogen atoms.

The terms “polycyclyl” or “polycyclic radical” include moieties with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminoacarbonyl, aralkylaminocarbonyl, alkenylaminocarbonyl, alkylcarbonyl, arylcarbonyl, aralkylcarbonyl, alkenylcarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

The term “heteroatom” includes atoms of any element other than carbon or hydrogen. Preferred heteroatoms are nitrogen, oxygen, sulfur and phosphorus.

The term “electron-withdrawing group” “or electron-withdrawing atom” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (Σ) constant. This well known constant is described in many references, for instance, J. March, Advanced Organic Chemistry, McGraw Hill Book Company, New York, (1977 edition) pp. 251-259. The Hammett constant values are generally negative for electron donating groups (Σ[P]=−0.66 for NH2) and positive for electron withdrawing groups (Σ[P]=0.78 for a nitro group), wherein Σ[P] indicates para substitution. Non-liminting examples of electron-withdrawing groups include nitro, acyl, formyl, sulfonyl, trifluoromethyl, cyano, chloride, carbonyl, thiocarbonyl, ester, imino, amido, carboxylic acid, sulfonic acid, sulfinic acid, sulfamic acid, phosphonic acid, boronic acid, sulfate ester, hydroxyl, mercapto, cyano, cyanate, thiocyanate, isocyanate, isothiocyanate, carbonate, nitrate and nitro groups and the like. Exemplary electron-withdrawing atoms include, but are not limited to, an oxygen atom, a nitrogen atom, a sulfur atom or a halogen atom, such as a fluorine, chlorine, bromine or iodine atom. It is to be understood that, unless otherwise indicated, reference herein to an acidic functional group also encompasses salts of that functional group in combination with a suitable cation.

Additionally, the phrase “any combination thereof” implies that any number of the listed functional groups and molecules may be combined to create a larger molecular architecture. For example, the terms “phenyl,” “carbonyl” (or “═O”), “—O—,” “—OH,” and C1-6 (i.e., —CH3 and —CH2CH2CH2-) can be combined to form a 3-methoxy-4-propoxybenzoic acid substituent. It is to be understood that when combining functional groups and molecules to create a larger molecular architecture, hydrogens can be removed or added, as required to satisfy the valence of each atom.

The description of the disclosure herein should be construed in congruity with the laws and principals of chemical bonding. For example, it may be necessary to remove a hydrogen atom in order accommodate a substitutent at any given location. Furthermore, it is to be understood that definitions of the variables (i.e., “R groups”), as well as the bond locations of the generic formulae of the invention (e.g., formulas I or II), will be consistent with the laws of chemical bonding known in the art. It is also to be understood that all of the compounds of the invention described above will further include bonds between adjacent atoms and/or hydrogens as required to satisfy the valence of each atom. That is, bonds and/or hydrogen atoms are added to provide the following number of total bonds to each of the following types of atoms: carbon: four bonds; nitrogen: three bonds; oxygen: two bonds; and sulfur: two-six bonds.

It will be noted that the structures of some of the compounds of this invention include asymmetric carbon atoms. It is to be understood accordingly that the isomers arising from such asymmetry (e.g., all enantiomers, stereoisomers, rotamers, tautomers, diastereomers, or racemates) are included within the scope of this invention. Such isomers can be obtained in substantially pure form by classical separation techniques and by stereochemically controlled synthesis. Furthermore, the structures and other compounds and moieties discussed in this application also include all tautomers thereof. Compounds described herein may be obtained through art recognized synthesis strategies.

It will also be noted that the substituents of some of the compounds of this invention include isomeric cyclic structures. It is to be understood accordingly that constitutional isomers of particular substituents are included within the scope of this invention, unless indicated otherwise. For example, the term “tetrazole” includes tetrazole, 2H-tetrazole, 3H-tetrazole, 4H-tetrazole and 5H-tetrazole.

The definitions of certain terms as used in this specification are provided below. Definitions of other terms may be found in the glossary provided by the U.S. Department of Energy, Office of Science, Human Genome Project. In practicing the present invention, many conventional techniques in molecular biology, microbiology and recombinant DNA are used. These techniques are well-known and are explained in, e.g., Current Protocols in Molecular Biology, Vols. I-III, Ausubel, ed. (1997); Sambrook et al., Molecular Cloning: A Laboratory Manual, Second Edition (Cold Spring Harbor Laboratory Press, Cold Spring Harbor, N.Y., 1989); DNA Cloning: A Practical Approach, Vols. I and II, Glover D, ed. (1985); Oligonucleotide Synthesis, Gait, ed. (1984); Nucleic Acid Hybridization, Hames & Higgins, Eds. (1985); Transcription and Translation, Hames & Higgins, eds. (1984); Animal Cell Culture, Freshney, ed. (1986); Immobilized Cells and Enzymes (IRL Press, 1986); Perbal, A Practical Guide to Molecular Cloning; the series, Methods in Enzymol. (Academic Press, Inc., 1984); Gene Transfer Vectors for Mammalian Cells, Miller and Calos, Eds. (Cold Spring Harbor Laboratory, NY, 1987); and Methods in Enzymology, Vols. 154 and 155, Wu and Grossman, and Wu, Eds., respectively.

As used herein a “reporter” gene is used interchangeably with the term “marker gene” and is a nucleic acid that is readily detectable and/or encodes a gene product that is readily detectable such as luciferase.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced and translated into the protein encoded by the coding sequence.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The phrases “therapeutically effective amount” and “effective amount” are used herein to mean an amount sufficient to reduce by at least about 15 percent, preferably by at least 50 percent, more preferably by at least 90 percent, and most preferably prevent, a clinically significant deficit in the activity, function and response of the host. Alternatively, a therapeutically effective amount is sufficient to cause an improvement in a clinically significant condition/symptom in the host.

“Agent” refers to all materials that may be used to prepare pharmaceutical and diagnostic compositions, or that may be compounds, nucleic acids (including inhibitory nucleic acids such as shRNA, RNAi, etc.), small molecules, polypeptides, fragments, isoforms, variants, or other materials that may be used independently for such purposes, all in accordance with the present invention.

“Modulator” as used herein can be any substance, including but not limited to a drug, a compound, a protein or a peptide, capable of enhancing or diminishing Axin stabilization, and thereby influence Wnt signaling. The modulator is able to interact with Axin directly or indirectly, in such a way that it may enhance or inhibit Wnt signaling.

“Derivative” refers to either a compound, a protein or polypeptide that comprises an amino acid sequence of a parent protein or polypeptide that has been altered by the introduction of amino acid residue substitutions, deletions or additions, or a nucleic acid or nucleotide that has been modified by either introduction of nucleotide substitutions or deletions, additions or mutations. The derivative nucleic acid, nucleotide, protein or polypeptide possesses a similar or identical function as the parent polypeptide.

The term “double-stranded RNA” or “dsRNA”, as used herein, refers to a complex of ribonucleic acid molecules, having a duplex structure comprising two anti-parallel and substantially complementary, as defined above, nucleic acid strands. The two strands forming the duplex structure may be different portions of one larger RNA molecule, or they may be separate RNA molecules. Where separate RNA molecules, such siRNA are often referred to in the literature as siRNA (“short interfering RNA”). Where the two strands are part of one larger molecule, and therefore are connected by an uninterrupted chain of nucleotides between the 3′-end of one strand and the 5′ end of the respective other strand forming the duplex structure, the connecting RNA chain is referred to as a “hairpin loop”, “short hairpin RNA” or “shRNA”. Where the two strands are connected covalently by means other than an uninterrupted chain of nucleotides between the 3′-end of one strand and the 5′ end of the respective other strand forming the duplex structure, the connecting structure is referred to as a “linker”. The RNA strands may have the same or a different number of nucleotides. The maximum number of base pairs is the number of nucleotides in the shortest strand of the siRNA minus any overhangs that are present in the duplex. In addition to the duplex structure, a siRNA may comprise one or more nucleotide overhangs. In addition, as used in this specification, “siRNA” may include chemical modifications to ribonucleotides, including substantial modifications at multiple nucleotides and including all types of modifications disclosed herein or known in the art. Any such modifications, as used in an siRNA type molecule, are encompassed by “siRNA” for the purposes of this specification and claims.

As used herein, a “nucleotide overhang” refers to the unpaired nucleotide or nucleotides that protrude from the duplex structure of a siRNA when a 3′-end of one strand of the siRNA extends beyond the 5′-end of the other strand, or vice versa. “Blunt” or “blunt end” means that there are no unpaired nucleotides at that end of the siRNA, i.e., no nucleotide overhang. A “blunt ended” siRNA is a siRNA that is double-stranded over its entire length, i.e., no nucleotide overhang at either end of the molecule. For clarity, chemical caps or non-nucleotide chemical moieties conjugated to the 3′ end or 5′ end of an siRNA are not considered in determining whether an siRNA has an overhang or is blunt ended.

The term “antisense strand” refers to the strand of a siRNA which includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches are most tolerated in the terminal regions and, if present, are generally in a terminal region or regions, e.g., within 6, 5, 4, 3, or 2 nucleotides of the 5′ and/or 3′ terminus.

The term “sense strand,” as used herein, refers to the strand of a siRNA that includes a region that is substantially complementary to a region of the antisense strand.

“Introducing into a cell”, when referring to a siRNA, means facilitating uptake or absorption into the cell, as is understood by those skilled in the art. Absorption or uptake of siRNA can occur through unaided diffusive or active cellular processes, or by auxiliary agents or devices. The meaning of this term is not limited to cells in vitro; a siRNA may also be “introduced into a cell”, wherein the cell is part of a living organism. In such instance, introduction into the cell will include the delivery to the organism. For example, for in vivo delivery, siRNA can be injected into a tissue site or administered systemically. In vitro introduction into a cell includes methods known in the art such as electroporation and lipofection.

The term “binding” refers to the physical association of a component (e.g., an Axin protein) with another component (e.g., an Axin-associated protein). A measurement of binding can lead to a value such as a dissociation constant, an association constant, on-rate or off-rate.

As used herein, the term “conditions permitting the binding.” refers to conditions of, for example, temperature, salt concentration, pH and protein concentration under which binding will arise. Exact binding conditions will vary depending upon the nature of the assay, for example, whether the assay uses pure proteins or only partially purified proteins. Temperatures for binding can vary from 15° C. to 37° C., but will preferably be between room temperature and about 30° C. The concentration of Axin in a binding reaction will also vary, but will preferably be about 10 pM to 10 nM (e.g., in a reaction using radiolabeled components).

As the term is used herein, binding is “specific” if it occurs with a Kd of 1 mM or less, generally in the range of 100 nM to 10 pM. For example, binding is specific if the Kd is 100 nM, 50 nM, 10 nM, 1 nM, 950 pM, 900 pM, 850 pM, 800 pM, 750 pM, 700 pM, 650 pM, 600 pM, 550 pM, 500 pM, 450 pM, 350 pM, 300 pM, 250 pM, 200 pM, 150 pM, 100 pM, 75 pM, 50 pM, 25 pM, 10 pM or less.

As used herein, “expression” includes but is not limited to one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “mutant” means any heritable variation from the wild-type that is the result of a mutation, e.g., single nucleotide polymorphism (“SNP”). The term “mutant” is used interchangeably with the terms “marker”, “biomarker”, and “target” throughout the specification.

### Wnt Signal Transduction Pathway

The Wnt gene family encodes a large class of secreted proteins related to the Int1/Wnt1 proto-oncogene and Drosophila wingless (“Wg”), a Drosophila Wnt1 homologue (Cadigan et al. (1997) Genes & Development 11:3286-3305). Wnts are expressed in a variety of tissues and organs and are required for many developmental processes, including segmentation in Drosophila; endoderm development in C. elegans; and establishment of limb polarity, neural crest differentiation, kidney morphogenesis, sex determination, and brain development in mammals (Parr, et al. (1994) Curr. Opinion Genetics & Devel. 4:523-528). The Wnt pathway is a master regulator in animal development, both during embryogenesis and in the mature organism (Eastman, et al. (1999) Curr Opin Cell Biol 11: 233-240; Peifer, et al. (2000) Science 287: 1606-1609).

Wnt signals are transduced by the Frizzled (“Fz”) family of seven transmembrane domain receptors (Bhanot et al. (1996) Nature 382:225-230). Wnt ligands bind to Fzd, and in so doing, activate the cytoplasmic protein Dishevelled (Dvl-1, 2 and 3 in humans and mice) (Boutros, et al. (1999) Mech Dev 83: 27-37) and phosphorylate LRP5/6. A signal is thereby generated which prevents the phosphorylation and degradation of Armadillo/β(beta)-catenin, in turn leading to the stabilization of β-catenin (Perrimon (1994) Cell 76:781-784). This stabilization is occasioned by Dvl's association with axin (Zeng et al. (1997) Cell 90:181-192), a scaffolding protein that brings various proteins together, including GSK3, APC, CK1, and β-catenin, to form the β-catenin destruction complex. The evolutionarily conserved canonical Wnt/β-catenin signal transduction cascade controls many aspects of metazoan development. Context-dependent activation of the pathway is involved in embryonic cell fate decisions, stem cell regulation and tissue homeostasis1. A key feature of the Wnt/β-catenin pathway is the regulated proteolysis of the downstream effector β-catenin by the β-catenin destruction complex. The principal constituents of the β-catenin destruction complex are adenomatous polyposis coli (APC), Axin, and GSK3α/β. In the absence of Wnt pathway activation, cytosolic β-catenin is constitutively phosphorylated and targeted for degradation. Upon Wnt stimulation, the β-catenin destruction complex disassociates, which leads to the accumulation of nuclear β-catenin and transcription of WNT pathway responsive genes.

Inappropriate activation of the pathway, mediated by overexpression of Wnt proteins or mutations affecting components of the β-catenin destruction complex, has been observed in many cancers (Polakis, P. (2007) Curr Opin Genet Dev 17, 45-51). Notably, truncating mutations of the tumor suppressor APC are the most prevalent genetic alterations in colorectal carcinomas. In addition, Axin1 and Axin2 mutations have been identified in patients with hepatocarcinomas and colorectal cancer respectively. (Taniguchi, K. et al. (2002) Oncogene 21, 4863-71; Liu, W. et al. (2000) Nat Genet. 26, 146-7; Lammi, L. et al. (2004) Am J Hum Genet. 74). These somatic mutations result in Wnt-independent stabilization of β-catenin and constitutive activation of β-catenin-mediated transcription.

Aberrant Wnt pathway activation, through the stabilization of β-catenin, plays a central role in tumorigenesis for many colorectal carcinomas. It is estimated that 80% of colorectal carcinomas (CRCs) harbor inactivating mutations in the tumor repressor APC, which allows for uninterrupted Wnt signaling. Furthermore, there is a growing body of evidence that suggests that Wnt-pathway activation may be involved in melanoma, breast, liver, lung, and gastric cancers. There is a long-recognized connection between Wnts, normal development, and cancer, a connection further established with the identification the c-Myc proto-oncogene as a target of Wnt signaling (He et al. (1998) Science 281:1509-3512).

Furthermore, other disorders are associated with aberrant Wnt signaling, include but are not limited to osteoporosis, osteoarthritis, polycystic kidney disease, diabetes, schizophrenia, vascular disease, cardiac disease, non-oncogenic proliferative diseases, and neurodegenerative diseases such as Alzheimer's disease.

### Axin

Axin is a key regulator of Wnt signaling, acting to marshal together the protein components of the β-catenin destruction complex (GSK3, APC, CK1, and β-catenin). Glycogen synthase kinase 3 (GSK3, known as shaggy in Drosophila), the tumor suppressor gene product APC (adenomatous polyposis coli) (Gumbiner (1997) Curr. Biol. 7:R443-436), and Axin, are all negative regulators of the Wnt pathway. In the absence of a Wnt ligand, these proteins form the β-catenin destruction complex and promote phosphorylation and degradation of β-catenin, whereas Wnt signaling inactivates the complex and prevents β-catenin degradation. Stabilized β-catenin translocates to the nucleus as a result, where it binds TCF (T cell factor) transcription factors (also known as lymphoid enhancer-binding factor-1 (LEF1)) and serves as a coactivator of TCF/LEF-induced transcription (Bienz, et al. (2000) Cell 103: 311-320; Polakis, et al. (2000) Genes Dev 14: 1837-1851).

The efficient assembly of the multi-protein destruction complex is dependent on the steady state levels of its principal constituents. Axin has been reported to be the concentration-limiting factor in regulating the efficiency of the β-catenin destruction complex, and increased expression of Axin can enhance β-catenin degradation in cell lines expressing truncated APC. (Salic, A., et al. (2000) Mol Cell 5, 523-32; Lee, E., et al. (2003) PLoS Biol 1, E10; Behrens, J., et al. (1998) Science 280, 596-9; Kishida, M., et al. (1999) Oncogene 18, 979-85). Thus, it is likely that Axin protein levels need to be tightly regulated to ensure proper WNT pathway signaling. For this reason, Tankyrase inhibitors such as XAV939 are such effective modulators of Wnt pathwasy signaling.

As described herein, chemical genetic and proteomic approaches were employed to search for novel modulators of the Wnt signaling pathway. As described, shown in the figures, and described experimentally herein, Axin stabilization is a robust mechanism through which to modulate Wnt signaling. Low molecular weight compounds were identified that can prolong the half-life of Axin and promote β-catenin degradation through inhibiting Tankyrase (TNKS). Furthermore, a novel mechanism was revealed that controls Axin protein stability, whose therapeutic exploitation holds promise for treating WNT pathway dependent cancers.

The human Axin gene encodes a 900-amino acid polypeptide with 87% identity to the mouse protein (known as “fused” (fu), and shown to cause axis duplication in homozygous mouse embryos). The sequence also contains a regulator of G protein signaling domain (RGS domain, which binds APC), a GSK3 binding domain, a β-catenin binding domain, a DIX domain (involved in self oligomerization), and a C-terminal region with homology to a conserved sequence near the N terminus of Drosophila and vertebrate ‘dishevelled’ proteins. And although the sequence contains a bipartite nuclear localization signal, Axin is not known to localize to the nucleus. (Zeng, et al. (1997) Cell 90: 181).

A small N-terminal region of Axin1 (amino acid 19-30), which encompasses the most conserved stretch of amino acids within Axin, was found to be both required and sufficient to interact with Tankyrase, as described herein. The specific interaction of Axin1 with TNKS1 through this small N-terminal domain, referred to herein as TBD (Tankyrase-Binding Domain), was further substantiated by GST pull-down and co-immunoprecipitation assays.

Axin exists as one of at least two forms, Axin1 and Axin2 (also called Axil, or conductin, in non-human species). Axin1 and Axin2 proteins have roughly 45% amino acid identity and essentially identical functions in regulating β-catenin levels. Unlike Axin2, however, Axin1 is not thought to be a β-catenin-TCF-regulated gene. Furthermore, Axin2's function in a feedback repressor pathway regulating Wnt signaling contributes to a belief that there may be potential functional differences between the effects of Wnt pathway activation on Axin1 vs. Axin2.

The compounds of the invention act as Axin stabilizers, as demonstrated experimentally throughout the present application. Said compounds increase the protein levels of Axin. Compounds that were identified as Wnt antagonists through a variety of assays were found to act via Axin stabilization. The discovery and validation of this mechanism gave rise to the methods of the present invention.

The compounds of the invention were found to inhibit Wnt signaling in a number of small molecule black box screenings. One screen was performed using SW480 cells (a colon cancer cell line with APC truncation) stably transfected with SuperTopflash, a TCF luciferase reporter. SW480 is a human colon carcinoma line that is APC deficient and characterized by constitutive, ligand-independent Wnt signaling. The signaling results from abnormal accumulation of stable β-catenin in the nucleus, since the β-catenin is not phosphorylated and degraded by the β-catenin destruction complex as in normal cells.

Additionally, the compounds of the invention were found to inhibit Wnt signaling in cell lines with an intact Wnt signaling pathway (e.g., 293T cells). Another screen was performed using 293T-STF cells treated with Wnt3a conditioned medium. In this screen, compounds were found to stabilize Axin in cells without active Wnt signaling (293T cells). This and use of inhibitory agents (e.g., RNAi and Wnt inhibitor proteins) found to block Wnt signaling a/different levels without concomitant Axin stabilization, leads to belief that the action of the Axin stabilizers of the invention is not the result of Wnt inhibition itself.

As described herein, the Axin stabilizers of the invention (e.g., the compounds of the invention) induce phosphorylation and degradation of β-catenin in colon cancer cells (e.g., SW480 cells) through a GSK3-dependent mechanism. Said stabilizers inhibit growth of colon cancer cells in in vitro cell culture assays. In one embodiment of the invention, said stabilizers increase phosphorylation of Axin by GSK3, which in turn stabilizes Axin and increases the interaction between Axin and β-catenin. This leads to accelerated phosphorylation and degradation of β-catenin.

The catalytic activity of TNKS is linked to the stability of Axin, and the Axin and TNKS have been shown to bond one another in coimmunoprecipitation experiments and in the yeast two-hybrid system.

### Tankyrase (TNKS)

“Tankyrase,” short for TRF1-interacting ankyrin related ADP-ribose polymerase, is a molecular scaffolding protein that possesses PARsylation activity. It is known to regulate vesicular trafficking (e.g., targeted delivery of newly synthesized proteins) based on its localization to the Golgi in non-polarized cells. (Yeh, et al. (2006) Biochem. J. 399:415). TNKS can also be found at telomeres, centrosomes, and nuclear pores. TNKS plays an essential regulatory role in mitotic segregation, and regulates telomere homeostasis by modifying the negative regulator of telomere length, TRF1. (Smith, et al. (1998) Science 282:1484) (Dynek, et al. (2004) Science 304:97).

TNKS1 and 2 are proteins comprising 1,327 and 1,166 residues, respectively. They are also referred to as PARP-5a and -5b, respectively. The proteins share about 83% sequence identity, and differ mainly in the absence of a histidine/praline/serine-rich (HPS) domain present only in TNKS1. Both proteins possess 24 ankyrin-type repeats for substrate binding, a sterile alpha motif (SAM) domain, involved in self oligomerization, and a C-terminal poly(ADP-ribose) polymerase (PARP) homology domain for catalytic activities. Critical residues required for NAD+ binding and catalysis are entirely conserved between the two proteins. Binding partners include IRAP (implicated in insulin signaling), Grb14 (implicated in insulin signaling), NuMA (implicated in cell cycle), and Mcl-1 (implicated in apoptosis).

Yeast two-hybrid assays described herein reveal that the region spanning III, IV, and V ankyrin repeat domains of TNKS1 is required and sufficient for its interaction with Axin1. Furtrhermore, β-catenin stabilization was found to require the Axin binding domain and the SAM domain, but not the PARP domain of TNKS1.

TNKS1 and TNKS2 function redundantly in regulating Axin protein levels. As demonstrated in at least SW480, HEK293, and DLD-1 cells (described herein), TNKS1 and TNKS2 need to be co-depeleted of in order to increase β-catenin phosphorylation, decrease β-catenin abundance, and inhibit the transcription of β-catenin target genes. Depletion of TNKS1 or TNKS2 alone does not lead to increased Axin1/2 protein levels.

TNKS1 and 2 belong to a family of NAD⁺-dependent enzymes called poly(ADP-ribose) polymerases, or PARPs, which modify themselves and other substrate proteins with ADP-ribose polymer. (Schreiber, et al. (2006) Nature Reviews Molecular Cell Biology; 7 (7):517). The addition of the ADP-ribose polymer (also known as PARsylation, or poly(ADP-ribose)ation) is a post-translational modification that regulates cell survival and cell-death functions, transcriptional regulation, telomere cohesion and mitotic spindle formation during cell division, energy metabolism, and intracellular trafficking.

In several cases, parsylation of a target protein has been linked to ubiquitin dependent degradation. For instance, parsylation of TRF1 by TNKS1 dissociates TRF1 from telomere and promotes its degradation. (Smith S, et al. (1998) Science; 282(5393):1484). Also, autoparsylation of TNKS promotes degradation of TNKS. (Yeh T Y J, et al. (2006) Biochemical Journal; 399:415).

As demonstrated experimentally through an siRNA-rescue approach described herein, the catalytic (PARsylation) activity of Tankyrase is required for the regulation of Axin protein levels and Wnt pathway signaling. The inhibition of said catalytic activity by Tankyrase inhibitors such as XAV939 results in Axin stability, as well as subsequent β-catenin degradation and cessation of Wnt signaling. Tankyrase inhibitors such as XAV939 in fact tightly bind to TNKS1/2 at their catalytic PARP domains. Tankyrase inhibitors such as XAV939 also hinder the auto-PARsylation ability of TNKS1/2, and can in fact increase TNKS protein levels while at the same time abrogating their catalytic functions.

### Tankyrase (TNKS) Inhibition and Wnt Signaling Inhibition

Through co-immunoprecipitation experiments described herein, TNKS1/2 were found to associate with Axin2 in SW480 cells, and through yeast two-hybrid assay experiments described herein, strong binding between Axin1/2 and TNKS1/2 is demonstrated. Physical interaction between Axin and TNKS, as mediated by the evolutionary conserved “Tankyrase Binding Domain” (TBD) in Axin, is critical for regulating Axin protein levels in vivo. As demonstrated herein (e.g., through siRNAs experiments), TNKS1/2 are the only PARP family members to affect Axin stability.

As described herein, Tankyrase inhibitors (e.g., XAV939) increase GSK3β-Axin complex formation and thereby promote the GSK3β-dependent phosphorylation and proteasomal degradation of β-catenin. This occurs even in cells with impaired APC function (e.g., colorectal cell line SW480, which harbors a truncated APC allele), as Tankyrase inhibitors (e.g., XAV939) can rescue the cells' otherwise defective ability to degrade β-catenin. Tankyrase inhibitors such as XAV939 physically interact with TNKS1/2 (as shown herein, e.g., in a fluorescence polarization assay), and are able to function both upstream and at the level of the β-catenin destruction complex; they engender an increase in Axin protein levels, without a corresponding increase in Axin transcript level.

As described in greater detail, shown in the figures, and demonstrated experimentally herein, TNKS1/2 are revealed to be efficacy targets for Axin stabilizers (including, e,g, Axin-stabilizing small molecules, inhibitory nucleic acids, and fusion proteins). Compounds which bind and inhibit the catalytic activity of TNKS1/2, and siRNAs against TNKS1/2, stabilize Axin while promoting the phosphorylation and degradation of β-catenin.

TNKS antagonists preferably act by reducing or inhibiting the catalytic activity of TNKS proteins (e.g., their ability to PARsylate target proteins such as Axin, as well as their ability to autoparsylate), and not by reducing TNKS protein or transcript levels. As experimentally described herein, TNKS physically associates with Axin and requires its PARsylation activity for the regulation of Axin protein levels. TNKS promotes the ubiquitination and degradation of Axin, which may be mediated, at least in part, through the direct PARsylation of Axin or components of the ubiquitin-proteosome pathway.

In short, Tankyrase inhibitors such as XAV939 increase Axin protein levels, increase phospho-β-catenin, decrease cytosolic β-catenin, and impact β-catenin target genes in a fashion analogous to β-catenin siRNA.

### Screening Assays

The invention provides methods (also referred to herein as a “screening assays”) for identifying modulators, i.e., candidate or test compounds or agents (e.g., peptides, peptidomimetics, small molecules or other drugs) which modulate Wnt signal transduction, e.g., via Axin stabilization and/or abrogation of TNKS catalytic activity. In one embodiment, said screening methods identify agents capable of modulating Axin stabilization and/or TNKS catalytic activity, which in turn are capable of modulating Wnt pathway signaling. Conversely, an Axin destabilizer discovered through the methods of the invention can be used to propogate, enhance, or otherwise agonize Wnt signaling.

Modulators of Wnt (e.g., modulators of Axin stabilization, modulators of TNKS) can include, for example, agonists and/or antagonists, and can include small molecules (e.g., the compounds of the invention), inhibitory nucleic acids, and fusion proteins. Examples of using of methods of the invention are described in detail in the Examples section of the present invention.

The term “agonist,” or “mimetic” of Wnt signaling, as used herein, is meant to refer to an agent that has an agonizing effect on TNKS (e.g., enhances the catalytic properties of TNKS), and/or an destabilizing effect on Axin, and therefore mimics or upregulates (e.g., potentiates or supplements) Wnt signaling. Said Wnt agonist inhibits, decreases or suppresses an Axin bioactivity (such as its ability to ubiquitinate and degrade β-catenin), and/or agonizes a TKNS activity (such as its PARsylation ability), and/or otherwise leads to Axin destabilization. “Mimetic” and “agonist” include but are not limited to a polypeptide, a peptide, a lipid, a carbohydrate, a nucleotide, and a small organic molecule. Candidate mimetics can be natural or synthetic compounds, including, for example, synthetic small molecules, compounds contained in extracts of animal, plant, bacterial or fungal cells, as well as conditioned medium from such cells.

A Wnt agonist may be capable of disrupting a binding event or complex formation between an Axin protein and other Wnt signaling proteins with which it normally associates (e.g., GSK3, APC, Dvl)(i.e., a Wnt agonists disrupts the β-catenin destruction complex). Said agonist is capable of contributing to β-cat stabilization and propagation or facilitation of Wnt signal transduction. Alternatively, a Wnt agonist can be a compound or agent that enhances TNKS catalytic activity.

The term “antagonist” or “inhibitor” of Wnt signaling, as used herein, is meant to refer to an agent that inhibits, arrests, or otherwise negatively regulates Wnt signaling, due to its stabilizing effect on Axin and/or its antagonizing effect on TNKS. Said Wnt antagonist can be a compound or agent which abrogates the catalytic (e.g., PARsylation) activity of a TNKS protein, or mimics a bioactivity of an Axin protein (e.g., forming the β-catenin destruction complex). “Inhibitors” and “antagonists” may be agents that decrease, block, or prevent, signaling (e.g., Wnt signaling) via a pathway and/or which prevent the formation of protein interactions and complexes.

In one embodiment, the invention provides assays for screening candidate or test compounds which bind to or modulate the activity of an Axin and/or TNKS protein or polypeptide or biologically active portion thereof. By way of example, the invention provides assays for screening candidate or test compounds or agents which are capable of modulating Axin and/or TNKS stabilization.

In another embodiment, the invention provides assays for Axin protein stability and/or levels screening, which can be used as primary or secondary (counterscreen) assay. For example, a luciferase reporter can be employed as part of primary screen, followed by an Axin protein stability and/or levels screen as counterscreen. Axin fusion proteins such as Axin GFP, Axin-Luciferase, Axin-Renilla, etc., can be generated and expressed in cells, and then treated with compounds to see if Axin is stabilized.

In another embodiment, Axin fusion proteins such as Axin GFP, Axin-Luciferase, Axin-Renilla, etc., can be generated and used in in vitro Axin degradation assays. Said assays employ extracts from cultured cells, tissues or embryos, which are in turn treated with compounds to see if the Axin fusion protein levels are affected.

Specific examples of screening assays for small molecule inhibitors of the Wnt/β-catenin pathway are described herein (e.g., using a Wnt-responsive Super-Topflash (STF) luciferase reporter assay in HEK293 cells), e.g., in the Examples section.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al., (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a Wnt receptor on its surface (e.g., Fzd) is contacted with a test agent and the ability of the test agent to modulate Wnt signaling is determined (e.g., by measuring an alteration in Axin and/or TNKS protein levels, or in Axin's and/or TNKS' association with Axin-associated proteins). In another embodiment, the ability of the test agent to modulate Wnt signaling is determined by, e.g., measuring an alteration in Axin phosphorylation by GSK3 (e.g., through use of a phospho-specific anti-Axin antibody). In yet another embodiment, the ability of the test agent to modulate Wnt signaling is determined by, e.g., measuring phosphorylation and degradation of β-catenin (and/or any alteration thereof).

By way of non-limiting example, an agent capable of inhibiting Wnt signaling, as discovered through use of the methods of the present invention, will exhibit an ability to stabilize Axin and/or antagonize TNKS. Said stabilization will be manifested as, e.g., a decrease in total β-catenin levels, and/or an increase in phospho-β-catenin levels (i.e., phosphorylated β-catenin). Said stabilization will also be manifested as, e.g., increasing Axin protein levels, decreasing TNKS catalytic activity, and/or increasing formation of the Axin-GSK3 complex.

The cell, for example, can be of mammalian origin or a yeast cell. Determining the ability of the test agent to bind to an Axin and/or TNKS protein can be accomplished, for example, by coupling the test agent with a radioisotope or enzymatic label such that binding of the test compound to an Axin protein can be determined by detecting the labeled agent in a complex. For example, test agents can be labeled with ¹²⁵I, ³⁵S, ¹⁴C, or ³H, either directly or indirectly, and the radioisotope detected by direct counting of radioemmission or by scintillation counting. Alternatively, test agents can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a test agent to modulate Wnt signaling (e.g., to interact with an Axin and/or TNKS protein) without the labeling of any of the interactants. For example, a microphysiometer can be used to detect the interaction of a test agent with an Axin or TNKS protein without the labeling of either the test agent or the protein. (McConnell, H. M. et al. (1992) Science 257:1906-1912). As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between ligand and receptor, between TNKS and TNKS-associated proteins, or between Axin and Axin-associated proteins.

In one embodiment, the assay comprises contacting a cell in which an Axin and/or TNKS protein is expressed with a protein known to associate with Axin and/or TNKS under normal conditions (e.g., an Axin-associated protein, as defined herein), or biologically-active portion thereof, to form an assay mixture; contacting the assay mixture with a test agent; and determining the ability of the test agent to interact with an Axin and/or TNKS protein, wherein determining said interaction comprises determining the ability of the test agent to disrupt the binding event between said Axin protein and said Axin-associated proteins, or biologically-active portions thereof. The disruption of the normal Axin: Axin-associated protein binding event, and/or the TNKS: TNKS-associated protein binding event, can be measured by an alteration in β-catenin phosphorylation as compared to the normal state (i.e., that in which there is no test agent).

In another embodiment, an assay is a cell-based assay comprising contacting a cell expressing an Axin target molecule (e.g., β-cat) and/or TNKS target molecule with a test agent and determining the ability of the test agent to modulate (e.g. stimulate or inhibit) the activity of the Axin and/or TNKS target molecule. Determining the ability of the test compound to modulate the activity of an Axin and/or TNKS target molecule can be accomplished; for example, by comparing β-cat phosphorylation levels in both the presence and absence of the test agent.

Determining the ability of the Axin and/or TNKS protein to bind to or interact with an Axin and/or TNKS target molecule and/or Axin-associated protein can be accomplished by one of the methods described above for determining direct binding. In one embodiment, determining the ability of the Axin and/or TNKS protein to bind to or interact with an Axin and/or TNKS target molecule and/or Axin-associated protein can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a regulatory element operatively linked to a nucleic acid encoding a detectable marker, e.g., luciferase), or detecting a cellular response, for example, development, differentiation, or rate of proliferation.

In yet another embodiment, an assay of the present invention is a cell-free assay in which an Axin and/or TNKS protein or biologically active portion thereof is contacted with a test compound and the ability of the test compound to bind to the Axin and/or TNKS protein or biologically active portion thereof is determined. Binding of the test compound to the Axin and/or TNKS protein can be determined either directly or indirectly as described above. In a preferred embodiment, the assay includes contacting the Axin and/or TNKS protein or biologically active portion thereof with a known compound which binds Axin and/or TNKS to form an assay mixture, contacting the assay mixture with a test compound, and determining the ability of the test compound to interact with an Axin and/or TNKS protein, wherein determining the ability of the test compound to interact with an Axin and/or TNKS protein comprises determining the ability of the test compound to preferentially bind to an Axin and/or TNKS protein or biologically active portion thereof as compared to the known compound.

In another embodiment, the assay is a cell-free assay in which an Axin and/or TNKS protein or biologically active portion thereof is contacted with a test compound and the ability of the test compound to modulate (e.g., stimulate or inhibit) the activity of the Axin and/or TNKS protein or biologically active portion thereof is determined. Determining the ability of the test compound to modulate the activity of an Axin protein can be accomplished, for example, by determining the ability of the Axin protein to bind to a target molecule or interact with an Axin-associated and/or TNKS-associated protein by one of the methods described above for determining direct binding. Determining the ability of the Axin and/or TNKS protein to bind to a target molecule can also be accomplished using a technology such as real-time Biomolocular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore™). Changes in the optical phenomenon surface plasmon resonance (SPR) can be used as an indication of real-time reactions between biological molecules.

In an alternative embodiment, determining the ability of the test compound to modulate the activity of an Axin and/or TNKS protein can be accomplished by determining the ability of the Axin and/or TNKS protein to further modulate the activity of a target molecule or Axin-associated and/or TNKS-associated protein. For example, the catalytic/enzymatic activity of the target molecule on an appropriate substrate can be determined as previously described.

In yet another embodiment, the cell-free assay involves contacting an Axin and/or TNKS protein or biologically active portion thereof with a known compound which binds the Axin and/or TNKS protein to form an assay mixture, contacting the assay mixture with a test compound, and determining the ability of the test compound to interact with the Axin and/or TNKS protein, wherein determining the ability of the test compound to interact with the Axin and/or TNKS protein comprises determining the ability of the Axin protein to preferentially bind to or modulate the activity of a target molecule or Axin-associated and/or TNKS-associated protein.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with upstream or downstream elements.

Accordingly, in an exemplary screening assay of the present invention, the compound of interest is contacted with an Axin and/or TNKS protein or binding partner, e.g., an Axin-associated and/or TNKS-associated protein. To the mixture of the compound and the Axin protein or Axin binding partner is then added a composition containing an Axin and/or TNKS binding partner or an Axin and/or TNKS protein, respectively. Detection and quantification of complexes of Axin proteins and Axin binding partners, and/or TNKS proteins and TNKS binding partners, provide a means for determining a compound's efficacy at inhibiting (or potentiating) complex formation between Axin and a binding partner. The efficacy of the compound can be assessed by generating dose response curves from data obtained using various concentrations of the test compound. Moreover, a control assay can also be performed to provide a baseline for comparison. In the control assay, isolated and purified Axin polypeptide or binding partner is added to a composition containing the Axin binding partner or Axin polypeptide, and the formation of a complex is quantitated in the absence of the test compound. Alternatively, in the control assay, isolated and purified TNKS polypeptide or binding partner is added to a composition containing the TNKS binding partner or TNKS polypeptide, and the formation of a complex is quantitated in the absence of the test compound.

The cell-free assays of the present invention are amenable to use of both soluble and/or membrane-bound forms of isolated proteins (e.g., Axin proteins or biologically active portions thereof or Axin-target molecules, and/or TNKS proteins or biologically active portions thereof or TNKS-target molecules). In the case of cell-free assays in which a membrane-bound form an isolated protein is used (e.g., a Axin and/or TNKS-target molecule or receptor) it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether).sub.n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In the cell based or cell-free assays described above, endogenous Axin1 and/or Axin2 levels can be measured by using Axin1 or Axin2 antibodies. Furthermore, Axin can be labeled with eptitope tags, to allow for measuring Axin protein levels in either cells or extracts.

In the cell based or cell-free assays described above, endogenous TNKS1 and/or TNKS2 levels can be measured by using TNKS1 or TNKS2 antibodies. Furthermore, Axin can be labeled with eptitope tags, to allow for measuring TNKS protein levels in either cells or extracts.

In more than one embodiment of the above assay methods of the present invention, it may be desirable to immobilize either Axin, an Axin-associated protein or a target molecule to facilitate separation of complexed from uncomplexed forms of one or both of the proteins, as well as to accommodate automation of the assay. In more than one embodiment of the above assay methods of the present invention, it may be desirable to immobilize either TNKS, a TNKS-associated protein or a target molecule to facilitate separation of complexed from uncomplexed forms of one or both of the proteins, as well as to accommodate automation of the assay. Binding of a test compound to an Axin and/or TNKS protein, or interaction of an Axin and/or TNKS protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix.

For example, glutathione-S-transferase/Axin fusion proteins or glutathione-5-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or Axin protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of Axin binding or activity determined using standard techniques.

Other techniques for immobilizing proteins on matrices can also be used in the screening assays of the invention. For example, either an Axin protein, Axin-associated protein, or an Axin-target molecule can be immobilized utilizing conjugation of biotin and streptavidin. By way of other example, either a TNKS protein, TNKS-associated protein, or a TNKS-target molecule can be immobilized utilizing conjugation of biotin and streptavidin. Biotinylated protein or target molecules can be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with Axin, Axin-associated proteins, or target molecules but which do not interfere with binding of the protein to its target molecule can be derivatized to the wells of the plate, and unbound target, Axin, or Axin-related protein trapped in the wells by antibody conjugation. Methods for detecting such complexes, in addition to those described above for the GST-immobilized complexes, include immunodetection of complexes using antibodies reactive with the Axin protein or target molecule, as well as enzyme-linked assays which rely on detecting an enzymatic activity associated with the Axin protein or target molecule.

In another embodiment, modulators of Axin and/or TNKS expression are identified in a method wherein a cell is contacted with a candidate compound and the expression of Axin and/or TNKS mRNA or protein in the cell is determined. The level of expression of mRNA or protein in the presence of the candidate compound is compared to the level of expression of mRNA or protein in the absence of the candidate compound. The candidate compound can then be identified as a modulator of Axin expression based on this comparison. For example, when expression of Axin and/or TNKS mRNA or protein is greater (statistically significantly greater) in the presence of the candidate compound than in its absence, the candidate compound is identified as a stimulator of Axin and/or TNKS mRNA or protein expression. Alternatively, when expression of Axin and/or TNKS mRNA or protein is less (statistically significantly less) in the presence of the candidate compound than in its absence, the candidate compound is identified as an inhibitor of Axin and/or TNKS mRNA or protein expression. The level of Axin and/or TNKS mRNA or protein expression in the cells can be determined by methods described herein for detecting Axin and/or TNKS mRNA or protein.

In yet another aspect of the invention, the Axin and/or TNKS proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al., (1993) Biotechniques 14:920-924; Iwabuchi et al. (1993) Oncogene 8:1693-1696; and Brent WO094/10300), to identify other proteins. which bind to or interact with Axin and/or TNKS proteins (“binding proteins” or “bp”) and modulate Axin and/or TNKS activity. Such binding proteins are also likely to be involved in the propagation of signals by the Axin and/or TNKS proteins as, for example, downstream elements of an Axin-mediated signaling pathway. Alternatively, such binding proteins are likely to be cell-surface molecules associated with non-Axin expressing cells, wherein such binding proteins are involved in signal transduction.

The two-hybrid system is based on the modular nature of most transcription factors, which consist of separable DNA-binding and activation domains. Briefly, the assay utilizes two different DNA constructs. In one construct, the gene that codes for an Axin protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an Axin-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the Axin protein.

This invention further pertains to novel agents identified by the above-described screening assays and to processes for producing such agents by use of these assays. Accordingly, in one embodiment, the present invention includes a compound or agent obtainable by a method comprising the steps of any one of the aformentioned screening assays (e.g., cell-based assays or cell-free assays). For example, in one embodiment, the invention includes a compound or agent obtainable by a method comprising contacting a cell which expresses a target molecule with a test compound and the determining the ability of the test compound to bind to, or modulate the activity of, the target molecule. In another embodiment, the invention includes a compound or agent obtainable by a method comprising contacting a cell which expresses a target molecule with an Axin and/or TNKS protein or biologically-active portion thereof, to form an assay mixture, contacting the assay mixture with a test compound, and determining the ability of the test compound to interact with, or modulate the activity of, the target molecule.

In another embodiment, the invention includes a compound or agent obtainable by a method comprising contacting an Axin and/or TNKS protein or biologically active portion thereof with a test compound and determining the ability of the test compound to bind to, or modulate (e.g., stimulate or inhibit) the activity of, the Axin and/or TNKS protein or biologically active portion thereof. In yet another embodiment; the present invention includes a compound or agent obtainable by a method comprising contacting an Axin and/or TNKS protein or biologically active portion thereof with a known compound which binds the Axin and/or TNKS protein to form an assay mixture, contacting the assay mixture with a test compound, and determining the ability of the test compound to interact with, or modulate the activity of the Axin and/or TNKS protein.

Accordingly, it is within the scope of this invention to further use an agent identified as described herein in an appropriate animal model. For example, an agent identified as described herein (e.g., an Axin and/or TNKS modulating agent, an antisense Axin and/or TNKS nucleic acid molecule, or an Axin and/or TNKS-binding partner) can be used in an animal model to determine the efficacy, toxicity, or side effects of treatment with such an agent. Alternatively, an agent identified as described herein can be used in an animal model to determine the mechanism of action of such an agent.

The present invention also pertains to uses of novel agents identified by the above-described screening assays for diagnoses, prognoses, and treatments as described herein. Accordingly, it is within the scope of the present invention to use such agents in the design, formulation, synthesis, manufacture, and/or production of a drug or pharmaceutical composition for use in diagnosis, prognosis, or treatment, as described herein. For example, in one embodiment, the present invention includes a method of synthesizing or producing a drug or pharmaceutical composition by reference to the structure and/or properties of a compound obtainable by one of the above-described screening assays.

For example, a drug or pharmaceutical composition can be synthesized based on the structure and/or properties of a compound obtained by a method in which a cell which expresses a target molecule (e.g., a protein downstream of Axin, e.g., β-cat) is contacted with a test compound and the ability of the test compound to bind to, or modulate the activity of, the target molecule is determined. In another exemplary embodiment, the present invention includes a method of synthesizing or producing a drug or pharmaceutical composition based on the structure and/or properties of a compound obtainable by a method in which an Axin and/or TNKS protein or biologically active portion thereof is contacted with a test compound and the ability of the test compound to bind to, or modulate (e.g., stimulate or inhibit) the activity of, the Axin and/or TNKS protein or biologically active portion thereof is determined.

### Compounds of the Invention

The term “compounds of the invention” and like terminology, as defined further hereing, are used herein to describe compounds which can be used, for instance, to antagonize Wnt pathway signalling (e.g., via Axin stabilization and/or inhibition of TNKS catalytic activity). The compounds include but are not limited to XAV939.

The compounds also include pharmaceutically acceptable salts, enantiomers, stereoisomers, rotamers, tautomers, diastereomers, or racemates of the “compounds of the invention” and the like.

### Pharmaceutical Compositions

A composition as described herein may be a pharmaceutical composition. The invention provides for pharmaceutical compositions comprising Wnt signaling antagonists admixed with a physiologically compatible carrier. Said pharmaceutical compositions are suitable for administration to a warm-blooded animal, especially a human (or to cells or cell lines derived from a warm-blooded animal, especially a human), for the treatment, amelioration, diagnosis, or prevention of a Wnt signaling-related disorder.

In addition to the active ingredients, these pharmaceutical compositions may contain a significant amount of one or more inorganic or organic, solid or liquid, pharmaceutically acceptable carriers, and physiologically acceptable diluents (such as water, phosphate buffered saline, or saline), which can be used pharmaceutically.

The phrases “therapeutically effective amount” and “effective amount” are used herein to mean an amount sufficient to reduce by at least about 15 percent, preferably by at least 50 percent, more preferably by at least 90 percent, and most preferably prevent, a clinically significant deficit in the activity, function and response of the host. Alternatively, a therapeutically effective amount is sufficient to cause an improvement in a clinically significant condition/symptom in the host.

The effective amount can vary depending on such factors as the size and weight of the subject, the type of illness, or the particular compound of the invention. For example, the choice of the compound of the invention can affect what constitutes an “effective amount.” One of ordinary skill in the art would be able to study the factors contained herein and make the determination regarding the effective amount of the compounds of the invention without undue experimentation.

The regimen of administration can affect what constitutes an effective amount. The compound of the invention can be administered to the subject either prior to or after the onset of a Wnt signaling-related disorder. Further, several divided dosages, as well as staggered dosages, can be administered daily or sequentially, or the dose can be continuously infused, or can be a bolus injection. Further, the dosages of the compound(s) of the invention can be proportionally increased or decreased as indicated by the exigencies of the therapeutic or prophylactic situation.

The language “pharmaceutical preparation” or “pharmaceutical composition” includes preparations suitable for administration to mammals, e.g., humans. When the compounds of the present invention are administered as pharmaceuticals to mammals, e.g., humans, they can be given per se or as a pharmaceutical composition containing, for example, 0.1 to 99.5% (more preferably, 0.5 to 90%) of active ingredient in combination with a pharmaceutically acceptable carrier.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Suitable pharmaceutical carriers are described in “Remington's Pharmaceutical Sciences” by E. W. Martin.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, buccal, sublingual, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc., administration by injection, infusion or inhalation; topical by lotion or ointment; and rectal by suppositories. Oral and/or IV administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 1.0 to about 100 mg per kg per day. An effective amount is that amount treats a Wnt signaling-related disorder.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition.

### Synthetic Procedure

Compounds of the present invention are prepared from commonly available compounds using procedures known to those skilled in the art, including any one or more of the following conditions without limitation:

Within the scope of this text, only a readily removable group that is not a constituent of the particular desired end product of the compounds of the present invention is designated a “protecting group,” unless the context indicates otherwise. The protection of functional groups by such protecting groups, the protecting groups themselves, and their cleavage reactions are described for example in standard reference works, such as e.g., Science of Synthesis: Houben-Weyl Methods of Molecular Transformation. Georg Thieme Verlag, Stuttgart, Germany. 2005. 41627 pp. (URL: http://www.science-of-synthesis.com (Electronic Version, 48 Volumes)); J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide and Derivate” (Chemistry of Carbohydrates: Monosaccharides and Derivatives), Georg Thieme Verlag, Stuttgart 1974. A characteristic of protecting groups is that they can be removed readily (i.e., without the occurrence of undesired secondary reactions) for example by solvolysis, reduction, photolysis or alternatively under physiological conditions (e.g., by enzymatic cleavage).

Acid addition salts of the compounds of the invention are most suitably formed from pharmaceutically acceptable acids, and include for example those formed with inorganic acids e.g. hydrochloric, hydrobromic, sulphuric or phosphoric acids and organic acids e.g. succinic, malaeic, acetic or fumaric acid. Other non-pharmaceutically acceptable salts e.g. oxalates can be used for example in the isolation of the compounds of the invention, for laboratory use, or for subsequent conversion to a pharmaceutically acceptable acid addition salt. Also included within the scope of the invention are solvates and hydrates of the invention.

The conversion of a given compound salt to a desired compound salt is achieved by applying standard techniques, in which an aqueous solution of the given salt is treated with a solution of base e.g. sodium carbonate or potassium hydroxide, to liberate the free base which is then extracted into an appropriate solvent, such as ether. The free base is then separated from the aqueous portion, dried, and treated with the requisite acid to give the desired salt.

In vivo hydrolyzable esters or amides of certain compounds of the invention can be formed by treating those compounds having a free hydroxy or amino functionality with the acid chloride of the desired ester in the presence of a base in an inert solvent such as methylene chloride or chloroform. Suitable bases include triethylamine or pyridine. Conversely, compounds of the invention having a free carboxy group can be esterified using standard conditions which can include activation followed by treatment with the desired alcohol in the presence of a suitable base.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Particularly preferred salts are sodium, lysine and arginine salts of the compounds of the invention. Such salts can be formed by procedures well known and described in the art.

Other acids such as oxalic acid, which can not be considered pharmaceutically acceptable, can be useful in the preparation of salts useful as intermediates in obtaining a chemical compound of the invention and its pharmaceutically acceptable acid addition salt.

Metal salts of a chemical compound of the invention include alkali metal salts, such as the sodium salt of a chemical compound of the invention containing a carboxy group.

Mixtures of isomers obtainable according to the invention can be separated in a manner known per se into the individual isomers; diastereoisomers can be separated, for example, by partitioning between polyphasic solvent mixtures, recrystallisation and/or chromatographic separation, for example over silica gel or by, e.g., medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Intermediates and final products can be worked up and/or purified according to standard methods, e.g., using chromatographic methods, distribution methods, (re-) crystallization, and the like.

### General Process Conditions

The following applies in general to all processes mentioned throughout this disclosure.

The process steps to synthesize the compounds of the invention can be carried out under reaction conditions that are known per se, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g., in the H+ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described in Science of Synthesis: Houben-Weyl Methods of Molecular Transformation. Georg Thieme Verlag, Stuttgart, Germany. 2005.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofuran or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of those solvents, for example aqueous solutions, unless otherwise indicated in the description of the processes. Such solvent mixtures may also be used in working up, for example by chromatography or partitioning.

The compounds, including their salts, may also be obtained in the form of hydrates, or their crystals may, for example, include the solvent used for crystallization. Different crystalline forms may be present.

The invention relates also to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ.

### Prodrugs

This invention also encompasses pharmaceutical compositions containing, and methods of treating Wnt signaling-related disorders through administering, pharmaceutically acceptable prodrugs of compounds of the compounds of the invention. For example, compounds of the invention having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the invention. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

Any reference to a compound of the present invention is therefore to be understood as referring also to the corresponding pro-drugs of the compound of the present invention, as appropriate and expedient.

Fusion Proteins

The invention provides chimeric or fusion proteins. As used herein, a “chimeric protein” or “fusion protein” comprises all or part (preferably biologically active) of a polypeptide of the invention operably linked to a heterologous polypeptide (i.e., a polypeptide other than the same polypeptide of the invention). Within the fusion protein, the term “operably linked” is intended to indicate that the polypeptide of the invention and the heterologous polypeptide are fused in frame to each other. The heterologous polypeptide can be fused to the N terminus or C terminus of the polypeptide of the invention.

One useful fusion protein is a GST fusion protein in which the polypeptide of the invention is fused to the C terminus of GST sequences. Such fusion proteins can facilitate the purification of a recombinant polypeptide of the invention.

In another embodiment, the fusion protein contains a heterologous signal sequence at its N terminus. For example, the native signal sequence of a polypeptide of the invention can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide of the invention is fused to sequences derived from a member of the immunoglobulin protein family. The immunoglobulin fusion proteins of the invention can be incorporated into pharmaceutical compositions and administered to a subject to inhibit an interaction between a ligand (soluble or membrane bound) and a protein on the surface of a cell (receptor), to thereby suppress signal transduction in vivo. The immunoglobulin fusion protein can be used to affect the bioavailability of a cognate ligand of a polypeptide of the invention. Inhibition of ligand/receptor interaction may be useful therapeutically, both for treating proliferative and differentiative disorders and for modulating (e.g., promoting or inhibiting) cell survival. Moreover, the immunoglobulin fusion proteins of the invention can be used as immunogens to produce antibodies directed against a polypeptide of the invention in a subject, to purify ligands and in screening assays to identify molecules which inhibit the interaction of receptors with ligands.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in frame to the polypeptide of the invention.

RNAi

The invention provides small interfering ribonucleic acid sequences (siRNA), as well as compositions and methods for inhibiting the expression of the TNKS1/2 gene or other genes responsible for Axin stabilization in a cell or mammal using siRNA. The invention also provides compositions and methods for treating Wnt signaling-related disorders, including pathological conditions and diseases in a mammal caused by the aberrant expression of the TNKS1/2 genes or genes responsible for Axin stabilization, or caused by the aberrant signaling of pathways of which said genes are integral members, using siRNA. siRNA directs the sequence-specific degradation of mRNA through a process known as RNA interference (RNAi).

The siRNA of the invention comprises an RNA strand (the antisense strand) having a region which is less than 30 nucleotides in length, generally 19-24 nucleotides in length, and is substantially complementary to at least part of an mRNA transcript of the TNKS1/2 genes or other genes responsible for Axin stabilization. The use of these siRNAs enables the targeted degradation of mRNAs of genes that are implicated in, e.g., the Wnt signaling pathways.

The siRNA molecules according to the present invention mediate RNA interference (“RNAi”). The term “RNAi” is well known in the art and is commonly understood to mean the inhibition of one or more target genes in a cell by siRNA with a region which is complementary to the target gene. Various assays are known in the art to test siRNA for its ability to mediate RNAi (see for instance Elbashir et al., Methods 26 (2002), 199-213). The effect of the siRNA according to the present invention on gene expression will typically result in expression of the target gene being inhibited by at least 10%, 33%, 50%, 90%, 95% or 99% when compared to a cell not treated with the RNA molecules according to the present invention.

“siRNA” or “small-interfering ribonucleic acid” according to the invention has the meanings known in the art, including the following aspects. The siRNA consists of two strands of ribonucleotides which hybridize along a complementary region under physiological conditions. The strands are separate but they may be joined by a molecular linker in certain embodiments. The individual ribonucleotides may be unmodified naturally occurring ribonucleotides, unmodified naturally occurring deoxyribonucleotides or they may be chemically modified or synthetic as described elsewhere herein.

The siRNA molecules in accordance with the present invention comprise a double-stranded region which is substantially identical to a region of the mRNA of the target gene. A region with 100% identity to the corresponding sequence of the target gene is suitable. This state is referred to as “fully complementary.” However, the region may also contain one, two or three mismatches as compared to the corresponding region of the target gene, depending on the length of the region of the mRNA that is targeted, and as such may be not fully complementary. In an embodiment, the RNA molecules of the present invention specifically target one given gene. In order to only target the desired mRNA, the siRNA reagent may have 100% homology to the target mRNA and at least 2 mismatched nucleotides to all other genes present in the cell or organism. Methods to analyze and identify siRNAs with sufficient sequence identity in order to effectively inhibit expression of a specific target sequence are known in the art. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group).

Another factor affecting the efficiency of the RNAi reagent is the target region of the target gene. The region of a target gene effective for inhibition by the RNAi reagent may be determined by experimentation. A suitable mRNA target region would be the coding region. Also suitable are untranslated regions, such as the 5′-UTR, the 3′-UTR, and splice junctions. For instance, transfection assays as described in Elbashir S. M. et al, 2001 EMBO J., 20, 6877-6888 may be performed for this purpose. A number of other suitable assays and methods exist in the art which are well known to the skilled person.

The length of the region of the siRNA complementary to the target, in accordance with the present invention, may be from 10 to 100 nucleotides, 12 to 25 nucleotides, 14 to 22 nucleotides or 15, 16, 17 or 18 nucleotides. Where there are mismatches to the corresponding target region, the length of the complementary region is generally required to be somewhat longer.

Because the siRNA may carry overhanging ends (which may or may not be complementary to the target), or additional nucleotides complementary to itself but not the target gene, the total length of each separate strand of siRNA may be 10 to 100 nucleotides, 15 to 49 nucleotides, 17 to 30 nucleotides or 19 to 25 nucleotides.

The phrase “each strand is 49 nucleotides or less” means the total number of consecutive nucleotides in the strand, including all modified or unmodified nucleotides, but not including any chemical moieties which may be added to the 3′ or 5′ end of the strand. Short chemical moieties inserted into the strand are not counted, but a chemical linker designed to join two separate strands is not considered to create consecutive nucleotides.

The phrase “a 1 to 6 nucleotide overhang on at least one of the 5′ end or 3′ end” refers to the architecture of the complementary siRNA that forms from two separate strands under physiological conditions. If the terminal nucleotides are part of the double-stranded region of the siRNA, the siRNA is considered blunt ended. If one or more nucleotides are unpaired on an end, an overhang is created. The overhang length is measured by the number of overhanging nucleotides. The overhanging nucleotides can be either on the 5′ end or 3′ end of either strand.

The siRNA according to the present invention confer a high in vivo stability suitable for oral delivery by including at least one modified nucleotide in at least one of the strands. Thus the siRNA according to the present invention contains at least one modified or non-natural ribonucleotide. A lengthy description of many known chemical modifications are set out in published PCT patent application WO 200370918 and will not be repeated here. Suitable modifications for oral delivery are more specifically set out in the Examples and description herein. Suitable modifications include, but are not limited to modifications to the sugar moiety (i.e. the 2′ position of the sugar moiety, such as for instance 2′-O-(2-methoxyethyl) or 2′-MOE) (Martin et al., Helv. Chim. Acta, 1995, 78, 486-504) i.e., an alkoxyalkoxy group) or the base moiety (i.e. a non-natural or modified base which maintains ability to pair with another specific base in an alternate nucleotide chain). Other modifications include so-called ‘backbone’ modifications including, but not limited to, replacing the phosphoester group (connecting adjacent ribonucleotides with for instance phosphorothioates, chiral phosphorothioates or phosphorodithioates). Finally, end modifications sometimes referred to herein as 3′ caps or 5′ caps may be of significance. Caps may consist of more complex chemistries which are known to those skilled in the art.

In one embodiment, the invention provides double-stranded ribonucleic acid (dsRNA) molecules for inhibiting the expression of the TNKS1/2 genes or other genes responsible for Axin stabilization. The dsRNA comprises at least two sequences that, are complementary to each other. The dsRNA comprises a sense strand comprising a first sequence and an antisense strand comprising a second sequence. The antisense strand comprises a nucleotide sequence which is substantially complementary to at least part of an mRNA encoding TNKS1/2 genes or other genes responsible for Axin stabilization, and the region of complementarity is less than 30 nucleotides in length, generally 19-24 nucleotides in length. The dsRNA, upon contacting with a cell expressing the TNKS1/2 genes or other genes responsible for Axin stabilization, inhibits the expression of said genes by at least 40%.

In another embodiment, the invention provides a cell comprising one of the dsRNAs of the invention. The cell is generally a mammalian cell, such as a human cell.

In another embodiment, the invention provides a pharmaceutical composition for inhibiting the expression of the TNKS1/2 genes or other genes responsible for Axin stabilization in an organism, generally a human subject, comprising one or more of the dsRNA of the invention and a pharmaceutically acceptable carrier or delivery vehicle.

In another embodiment, the invention provides a method for inhibiting the expression of the TNKS1/2 genes or other genes responsible for Axin stabilization in a cell, comprising the following steps:

(a) introducing into the cell a double-stranded ribonucleic acid (dsRNA), wherein the dsRNA comprises at least two sequences that are complementary to each other. The dsRNA comprises a sense strand comprising a first sequence and an antisense strand comprising a second sequence. The antisense strand comprises a region of complementarity which is substantially complementary to at least a part of a mRNA encoding TNKS1/2 genes or other genes responsible for Axin stabilization, and wherein the region of complementarity is less than 30 nucleotides in length, generally 19-24 nucleotides in length, and wherein the dsRNA, upon contact with a cell expressing the TNKS1/2 genes or other genes responsible for Axin stabilization, inhibits expression of said genes by at least 40%; and

(b) maintaining the cell produced in step (a) for a time sufficient to obtain degradation of the mRNA transcript of the TNKS1/2 genes or other genes responsible for Axin stabilization, thereby inhibiting expression of said genes in the cell.

In another embodiment, the invention provides vectors for inhibiting the expression of the TNKS1/2 genes or other genes responsible for Axin stabilization in a cell, comprising a regulatory sequence operably linked to a nucleotide sequence that encodes at least one strand of one of the siRNA of the invention.

Inhibitory nucleic acid compounds of the present invention may be synthesized by conventional means on a commercially available automated DNA synthesizer, e.g. an Applied Biosystems (Foster City, Calif.) model 380B, 392 or 394 DNA/RNA synthesizer, or like instrument. Phosphoramidite chemistry may be employed. The inhibitory nucleic acid compounds of the present invention may also be modified, for instance, nuclease resistant backbones such as e.g., phosphorothioate, phosphorodithioate, phosphoramidate, or the like, described in many references may be used. The length of the inhibitory nucleic acid has to be sufficient to ensure that the biological activity is inhibited. Thus, for instance in case of antisense oligonucleotides, has to be sufficiently large to ensure that specific binding will take place only at the desired target polynucleotide and not at other fortuitous sites. The upper range of the length is determined by several factors, including the inconvenience and expense of synthesizing and purifying oligomers greater than about 30-40 nucleotides in length, the greater tolerance of longer oligonucleotides for mismatches than shorter oligonucleotides, and the like. Preferably, the antisense oligonucleotides of the invention have lengths in the range of about 15 to 40 nucleotides. More preferably, the oligonucleotide moieties have lengths in the range of about 18 to 25 nucleotides.

Double-stranded RNA, i.e., sense-antisense RNA, also termed small interfering RNA (siRNA) molecules, can also be used to inhibit the expression of nucleic acids for TNKS1/2 genes or other genes responsible for Axin stabilization. RNA interference is a method in which exogenous, short RNA duplexes are administered where one strand corresponds to the coding region of the target mRNA (Elbashir et al. (2001) Nature 411: 494). Upon entry into cells, siRNA molecules cause not only degradation of the exogenous RNA duplexes, but also of single-stranded RNAs having identical sequences, including endogenous messenger RNAs. Accordingly, siRNA may be more potent and effective than traditional antisense RNA methodologies since the technique is believed to act through a catalytic mechanism. Preferred siRNA molecules are typically from 19 to 25 nucleotides long, preferably about 21 nucleotides in length. Effective strategies for delivering siRNA to target cells include, for example, transduction using physical or chemical transfection.

Alternatively siRNAs may be expressed in cells using, e.g., various PolIII promoter expression cassettes that allow transcription of functional siRNA or precursors thereof. See, for example, Scherr et al. (2003) Curr. Med. Chem. 10(3):245; Turki et al. (2002) Hum. Gene Ther. 13(18):2197; Cornell et al. (2003) Nat. Struct. Biol. 10(2):91. The invention also covers other small RNAs capable of mediating RNA interference (RNAi) such as for instance micro-RNA (miRNA) and short hairpin RNA (shRNA).

The following Examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Example 1

### Screening Assay to Identify Small Molecule Wnt Inhibitors

To identify small molecule inhibitors of the Wnt/β-catenin pathway, a high-throughput compound screen was employed, with over one million compounds, using a Wnt-responsive Super-Topflash (STF) luciferase reporter assay in HEK293 cells. Based on its selectivity profile and potency, subsequent studies focused on a compound referred to herein as XAV939. XAV939 strongly inhibited Wnt3A stimulated STF activity in HEK293 cells but did not affect CRE, NF-κB, or TGFβ luciferase reporters. In contrast, LDW643, a close structural analogue of XAV939, had no activity on the Wnt3A induced STF reporter. XAV939 treatment was found to block Wnt3A-induced accumulation of β-catenin in HEK293 cells, indicating that the compound modulates WNT pathway activity upstream of β-catenin.

To test whether XAV939 functions upstream or at the level of the destruction complex to facilitate β-catenin degradation, the effect of compound treatment in the colorectal cancer cell line SW480 was tested. The SW480 cell line harbors a truncated APC allele and thereby has impaired destruction complex activity. Interestingly, XAV939 also inhibited STF activity in SW480 cells, albeit not to as great an extent as in HEK293 cells, which have an intact WNT pathway cascade. Consistent with this reduction in STF activity, XAV939 decreased β-catenin abundance but significantly increased β-catenin phosphorylation (S33/S37/T41) in SW480 cells, suggesting that XAV939 promotes the phosphorylation-dependent degradation of β-catenin. These findings indicate that XAV939 can restore β-catenin degradation even in cells with impaired APC function, possibly by modulating the activity of the destruction complex.

To explore how XAV939 may increase the activity of the destruction complex, the effects of compound treatment on protein levels of known WNT pathway components were studied. Strikingly, the protein levels of Axin1 and Axin2 were strongly increased after XAV939 treatment, whereas their transcript levels were unaffected by compound treatment. In addition, a strong increase in Axin-GSK3β complex formation was observed, presumably because of enhanced recruitment of GSK3β to the Axin complex in response to increased Axin protein levels. This phenomenon was confirmed by observing the effects of XAV939 on Axin1/2 protein levels, β-catenin degradation, and β-catenin target gene expression in DLD-1 cells, another colorectal cancer cell line with truncated APC.

Importantly, siRNA-mediated depletion of Axin1/2 in SW480 cells reversed the effect of XAV939 on β-catenin degradation and diminished the inhibitory activity of XAV939 on the STF reporter, further indicating that XAV939 inhibits WNT signaling by increasing Axin1/2 protein levels. Together, these findings demonstrate that XAV939 increases GSK3β-Axin complex formation and thereby promotes the GSK3β-dependent phosphorylation and proteasomal degradation of β-catenin.

The SuperTopFlash (STF) methods described in at least Example 1 employed plasmids manufactured as follows: SuperTopflash reporter was generated by inserting twelve TCF binding sites into pTA-Luc (Clontech). Mouse Axin1 and its mutants were fused with either GFP or FLAG epitope at the amino termini and cloned into a retroviral vector under the control of the metallothionein promoter. Human TNKS1/2 and their mutants were tagged with FLAG epitope fused at the amino termini and cloned into a mammalian expression vector under the control of the cytomegalovirus (CMV) promoter. Drosophila Axin fused with three HA epitopes at the carboxyl terminus was cloned in a Drosophila expression vector under the control of the metallothionein promoter. A sequence encoding the amino terminal fragment of mouse Axin1 (a.a. 1-87) was cloned in a Tet-regulated expression vector pcDNA4-TO (Invitrogen). Several proteins were cloned using Gateway technology (Invitrogen) into the expression vector pDEST15 (Invitrogen, Carlsbad, Calif.): TNKS1-P(1088-1327), TNKS2-P(934-1166), TNKS2-SP (872-1166), PARP1-P (662-1014) and PARP16-P (93-273).

### Example 3

### XAV939 Regulates Axin Protein Levels by Inhibiting Tankyrases

### Example 3a

### iTRAQ Quantitative Chemical Proteomics Approach

To identify the cellular efficacy target(s) through which XAV939 upregulates Axin protein levels, a 3-channel iTRAQ quantitative chemical proteomics approach was employed. This strategy is based on the immobilization of a bioactive analogue of XAV939 to affinity capture cellular proteins from HEK293 cell lysates. To discriminate specific binding from non-specific binding, a competition experiment was performed by spiking in an excess amount (20 μM) of the parental compound XAV939, the inactive analogue LDW643, or DMSO into the cell lysates prior to incubation with the immobilized compound. Specific binding to the immobilized compound, e.g. the presumed efficacy target(s) and potential off-targets, should be competed with XAV939 but not with LDW643.

By using iTRAQ, a chemical peptide labeling technique, the 3 samples were multiplexed and quantified binding displacement (% competition) relative to the vehicle (DMSO) by LC-MS/MS analysis. A total of 699 proteins were quantified. However, only 18 proteins were significantly and specifically competed-off (>65%, >2σ of the mean) with soluble XAV939, including the poly(ADP-ribose) polymerases PARP1 (93% competition), PARP2 (88% competition) PARP5a/TNKS1 (79% competition), and PARP5b/TNKS2 (74% competition). In addition, several protein modules containing known PARP1 substrates, such as the KU70 complex components (XRCC5, XRCC6) and the FACT components (SUPT16H, SSRP1) were significantly competed, presumably due to co-purification alongside PARP1. The majority of proteins were not competed (<2σ of the mean) suggesting that these are either highly abundant low affinity binders or proteins enriched on the affinity matrix by a binding mode that is distinct from binding to free compound in solution.

iTRAQ procedures were performed as described in Bantscheff et al (2007). In brief, gel lanes were cut into slices across the separation range and subjected to in-gel tryptic digestion, followed by iTRAQ labeling (Applied Biosystems.) Peptides extracted from the DMSO vehicle control was labeled with iTRAQ reagent 116 and combined with extracts from the compound-treated samples which were labeled with iTRAQ reagents 114 and 115 respectively. Sequencing was performed by liquid chromatography-tandem mass spectrometry on a LTQ-Orbitrap mass spectrometer (Thermo-Finnigan). Tandem mass spectra were generated using pulsed-Q dissociation, enabling detection of iTRAQ reporter ions. Peptide mass and fragmentation data were used to query an in-house curated version of the IPI database using Mascot (Matrix Science). Protein identifications were validated using a decoy database. iTRAQ reporter ion-based quantification was performed with in-house developed software.

### Example 3b

### Compound Competition

To establish the affinity of binding in vivo on the identified PARP proteins a dose response compound competition experiment was performed, which showed that XAV939 blocked TNKS binding at 0.1 μM and blocked PARP1/2 binding at 1 μM. As expected, the inactive compound LDW643 had no activity in this assay. The compound binding was further characterized using Cy5-labeled XAV939 and recombinant PARP proteins. XAV939 was found to tightly bind to the catalytic (PARP) domains of TNKS1 and TNKS2 (Kd 0.099 μM and 0.093 μM respectively). XAV939 also bound recombinant PARP1, although with lower binding affinity (Kd 1.2 μM).

To determine the affinity (equilibrium dissociation constant, Kd) for XAV939 with TNKS1, TNKS2 and PARP1, a titration of GST fusion proteins containing the PARP domain of TNKS1, TNKS2 or PARP1 with 50 nM XAV939 conjugated to Cy5 (XAV939^{Cy5}) in 50 mM Tris-HCl pH 8.0/50 mM NaCl/0.08% Triton X-100/10 mM MgCl2 was incubated at 30° C. for 2 hours in a black 384 well plate. Subsequently, fluorescence polarization (FP) values were obtained using optics optimized for Cy5 FP (optimized Cy5 FP Dual Emission Label, Perkin-Elmer) in a Perkin-Elmer Envision Plate Reader. Raw mP [1000×(S−G*P/S+G*P)] data were exported and analyzed with a one-site total binding saturation algorithm using GraphPad Prism.

### Example 3c

### siRNA Experiments

To determine which PARP family member(s) were the actual efficacy targets of XAV939-induced Axin protein accumulation, their siRNA-mediated loss-of function phenotypes were assessed. To circumvent potential redundancies among close family members, the two Tankyrase paralogs TNKS1 and TNKS2 were simultaneously targeted, as well as PARP1 and PARP2. Notably, co-depletion of TNKS1 and TNKS2 phenocopied the effect of XAV939 by increasing the protein levels of both Axin1 and Axin2, whereas PARP1/2 knockdown did not affect Axin protein levels. Together with the fact that XAV939 demonstrates higher affinity for TNKS1/2 than PARP1, these findings strongly suggest that TNKS1 and TNKS2 are the cellular efficacy targets of XAV939.

Additional siRNAs were used to further demonstrate that co-depletion of TNKS1 and TNKS2 increases β-catenin phosphorylation, decreases β-catenin abundance, and inhibits the transcription of β-catenin target genes in SW480 cells. Notably, depletion of TNKS1 or TNKS2 alone did not lead to increased Axin1/2 protein levels, indicating that TNKS1 and TNKS2 function redundantly in regulating Axin protein levels. Co-depletion of TNKS1 and TNKS2 also phenocopied XAV939 in HEK293 and DLD-1 cells.

Because many of the key Wnt pathway components are evolutionary conserved, the ability of TNKS to also regulates Axin protein levels and Wnt signaling in Drosophila cells was examined. A double-stranded RNA (dsRNA) targeting Drosophila TNKS increased the protein level, but not mRNA level, of exogenously expressed Drosophila Axin in S2 cells. In addition, TNKS dsRNA specifically inhibited a Wnt/Wingless reporter, but did not affect BMP or JAK/STAT pathway activity. These results support an evolutionarily conserved role for TNKS in regulating Axin protein levels and Wnt signaling.

TNKS1 and TNKS2 are members of the poly-(ADP-ribose) polymerase (PARP) family, which post-translationally modify their substrates through the addition of multiple ADP-ribose units, referred to as poly-ADP-ribosylation (PARsylation). An siRNA-rescue approach was employed to determine whether the PARsylation activity of TNKS is essential for regulating Axin protein levels. While depleting endogenous TNKS1/TNKS2, expression of exogenous siRNA-resistant wild-type TNKS2 or the catalytically inactive TNKS2-M1054V mutant was induced from a doxycyclin-responsive promoter. (Sbodio, J. I., et al. (2002) Biochem J 361, 451-9) Expression of wild-type, but not mutant TNKS2, rescued the effect of TNKS1/2 siRNAs on Axin1 protein expression, suggesting that the catalytic activity of Tankyrase is required for the regulation of Axin protein levels and WNT pathway signaling.

Sequences of siRNAs used in this study are shown as follows, in TABLE I:

TABLE I

| | | | |
|---|---|---|---|
| siRNA | Sense strand (5′ -> 3′) | Antisense strand (5′ -> 3′) | Supplier |

| | | | |
|---|---|---|---|
| TNKS1A | CUACAACAGAGUUCGAAUAUU | UAUUCGAACUCUGUUGUAGUU | Dharmacon |
| TNKS1B | GCAUGGAGCUUGUGUUAAUUU | AUUAACACAAGCUCCAUGCUU | Dharmacon |
| TNKS2A | GAGGGUAUCUCAUUAGGUAUU | UACCUAAUGAGAUACCCUCUU | Dharmacon |
| TNKS2B | GGAAAGACGUAGUUGAAUAUU | UAUUCAACUACGUCUUUCCUU | Dharmacon |
| AXIN1 | GGGCAUAUCUGGAUACCUGdTdT | CAGGUAUCCAGAUAUGCCCdTdT | Ambion |
| AXIN2 | GAGUAGCCAAAGCGAUCUAdTdT | UAGAUCGCUUUGGCUACUCdTdT | Qiagen |
| PARP1 | GAAAACAGGUAUUGGAUAUUU | AUAUCCAAUACCUGUUUUCUU | Dharmacon |
| PARP2 | AAGGAUUGCUUCAAGGUAAUU | UUACCUUGAAGCAAUCCUUUU | Dharmacon |
| PGL2 | CGUACGCGGAAUACUUCGAdTdT | UCGAAGUAUUCCGCGUACGdTdT | Dharmacon |
| CTNNB1 SP | GAUCCUAGCUAUCGUUCUUUU | AAGAACGAUAGCUAGGAUCUU | Dharmacon |
| | UAAUGAGGACCUAUACUUAUU | UAAGUAUAGGUCCUCAUUAUU | |
| | GCGUUUGGCUGAACCAUCAUU | UGAUGGUUCAGCCAAACGCUU | |
| | GGUACGAGCUGCUAUGUUCUU | GAACAUAGCAGCUCGUACCUU | |
| TNKS1 SP | CUACAACAGAGUUCGAAUAUU | UAUUCGAACUCUGUUGUAGUU | Dharmacon |
| | GCAUGGAGCUUGUGUUAAUUU | AUUAACACAAGCUCCAUGCUU | |
| | CGAAAGAGCCCAUAAUGAUUU | AUCAUUAUGGGCUCUUUCGUU | |
| | GAGAGUACACCUAUACGUAUU | UACGUAUAGGUGUACUCUCUU | |
| TNKS2 SP | GAGGGUAUCUCAUUAGGUAUU | UACCUAAUGAGAUACCCUCUU | Dharmacon |
| | GGAAAGACGUAGUUGAAUAUU | UAUUCAACUACGUCUUUCCUU | |
| | UAGCAUAACUCAAUUCGUAUU | UACGAAUUGAGUUAUGCUAUU | |
| | AGACAGAUCUUGUUACAUUUU | AAUGUAACAAGAUCUGUCUUU | |

### Example 3d

### Autoparsylation Activity Assay

Based on this findings above, the ability of XAV939 to regulate Axin protein levels by inhibiting the PARsylation activity of Tankyrases was examined. The C-terminal PARP domain of TNKS2 (GST-TNKS2^{PARP}) was efficiently auto-PARsylated using an in vitro PARsylation assay and this was fully inhibited by XAV939. In contrast, auto-PARsylation was not affected by the inactive control compound LDW643. Auto-PARsylation of TNKS has been reported to promote its degradation through the ubiquitin-proteasome pathway. (Yeh, T. Y. et al. (2006) Biochem J 399, 415-25) XAV939 treatment was found to significantly increase TNKS protein levels, suggesting that XAV939 also inhibits TNKS auto-PARsylation in vivo. Together, these genetic and biochemical analyses suggest that XAV939 increases Axin protein levels by inhibiting the catalytic activity of TNKS.

To assess the effect of compounds on auto-PARsylation of TNKS, the in vitro auto-PARsylation assay was perfomed as follows:

Tankyrase, utilizing NAD⁺, catalyzes poly(ADP-ribosyl)ation of itself (autoparsylation) or targeted proteins (substrate parsylations). In each reaction turnover, the enzyme consumes one unit of NAD⁺, add one unit of ADP-ribose to the polymer chain, releases one unit of nicotinamide. The autoparsylation activity assay is designed to monitor the nicotinamide formation and the reduction in the nicotinamide formation in the presence of tankyrase small molecule inhibitors. The quantification of nicotinamide is carried out by liquid chromatography/mass spectrometry (LC/MS). The assay is configured to 384-well format and is suitable for high throughput screen.

In general, the autoparsylation reactions were carried out in 40 μL volumes in the reaction solution containing the following: 5 μL compound (in 20% DMSO), 15 μl tankyrase in the Assay Buffer (50 mM Tris-HCl, pH 7.5, 10 mM MgCl₂, 50 mM NaCl, 1 mM DTT, 0.02% Tween-20, 8% Glycerol), 20 μL of NAD⁺. The final reaction mixture contains compound (inhibitor) with the concentration varying from 0.0086-18.75 μM, 2.5% DMSO, 20 nM GST-TNKS2P (or 60 nM GST-TNKS1P), 250 μM NAD⁺. All reactions were run at room temperature in 384-well Greiner flat-bottom plates (Costar, Cat. No. 781201) for 120 min then were quenched by the addition of 10 μL of 20% formic acid containing 500 nM d4-nicotinamide (CDN Isotopes, Inc. Cat. No. D3457). Prior to LC/MS analysis, the protein in the reaction mixture was removed by precipitation/centrifugation method after added two aliquots of acetonitrile. The obtained supernatant was then injected to LC/MS/MS system (Agilent 1200SL LC system, LEAP CTC HTC Autosampler and Sciex API 4000 mass spectrometer) where nicotinamide and the deuterated internal standard were retained by Hypercarb column (2.1×20 mm, 5 μM particle, Thermo Scientific Inc), gradient eluted and detected by the mass spectrometer that operated at the positive mode of electrospray ionization.

The LC was runs at 1 mL/min flow with the gradient from 5 to 95% acetonitrile in 0.8 min. 25 mM ammonium biocarbonate was added into the aqueous mobile phase and 0.1% ammonium hydroxide to the acetonitrile mobile phase. The Mass spectrometer runs in MRM mode and the mass transition for nicotinamide and d4-nicotinamide were 123→480 and 127→484, respectively. The relative responses (the ratio of nicotinamide produced from the enzymatic reaction to d4-nicotinamide, the internal standard) for the corresponding sample well were reported to assess the inhibitors activity or plotting the IC50 curves. Note: IC₅₀<0.0086 nM or IC₅₀>10 μM indicates the true IC₅₀ is out of experiment range. The proteins used in the tankyrase 1 and tankyrase 2 assays are truncated N-GST-tankyrase 1 (1088-1327) and truncated N-GST-tankyrase 2 (934-1166), respectively.

### Example 4

### Binding of TNKS to a Conserved N-Terminal Domain of Axin is Critical for Regulation of Axin Protein Levels

In order to explore how TNKS regulates Axin protein levels, co-immunoprecipitation experiments were performed, in which TNKS1 and TNKS2 were found to associate with Axin2 in SW480 cells. Additionally, strong binding between Axin1/2 and TNKS1/2 was detected in the yeast two-hybrid assay. The yeast two-hybrid assay was performed as follows:

The yeast two-hybrid assay was done using Matchmaker Two-Hybrid System 3 (Clontech) according to manufacture's instructions. Briefly, different fragments of mouse Axin1 were cloned into the bait plasmid pGBK-T7, and different fragments of human TNKS1 were cloned into prey plasmid pGAD-T7. AH109 cells were transformed with bait and prey plasmids. Double transformants were selected on Trp- and Leu-plates and examined for LacZ expression by 5-bromo-4-chloro-3-indolyl-³-D-galactopyranoside (X-gal) staining of a filter lift.

To define the TNKS binding domain in Axin1, various Axin1 fragments were tested for their ability to bind TNKS1. Strikingly, a small N-terminal region of Axin1 (amino acid 19-30), which encompasses the most conserved stretch of amino acids within Axin, was both required and sufficient to interact with TNKS1. The specific interaction of Axin1 with TNKS1 through this small N-terminal domain, herein named TBD (Tankyrase-Binding Domain), was further substantiated by GST pull-down and co-immunoprecipitation assays.

Inducible expression of the amino terminus Axin1 was achieved by stably transfecting pcDNA4-TO-Axin1 1-87 into HEK293-TRX cells (Invitrogen) and cells were induced by 10 ng/ml doxycyclin for 24 hours. Luciferase assays were performed with the Dual Luciferase Assay kit (Promega) according to the manufacturer's instructions.

The functional consequences of disrupting the physical interaction between Axin and TNKS were assessed. While cells expressing wild-type GFP-Axin1 demonstrated low basal protein levels that were strongly increased in response to XAV939 treatment, cells expressing GFP-Axin1Δ19-30 already exhibited high basal protein levels that did not further respond to compound treatment. Importantly, restoring the TNKS1-Axin1 interaction by fusing the heterologous TNKS binding domain of either IRAP or TRF1 to GFP-Axin1Δ19-30 fully restored its response to XAV939. These results demonstrate that overexpression of the N-terminal domain of Axin may compete with endogenous Axin for the binding of TNKS and thus increase the protein level of endogenous Axin1. Indeed, overexpression of GFP-Axin1N (a.a. 1-87), but not the mutant with the deleted TBD (lacking a.a. 19-30), substantially increased endogenous Axin1 protein levels while not affecting its mRNA expression. Together, these findings demonstrate that the physical interaction between Axin and TNKS, which is mediated by the evolutionary conserved TBD, is critical for regulating Axin protein levels in vivo.

Tankyrases contain ankryrin repeat domains for substrate binding, SAM domain for self-oligomerization, and PARP domain for catalytic activities. Using the yeast two-hybrid assay, we showed that the region spanning III, IV, and V ankyrin repeat domains of TNKS1 is required and sufficient for its interaction with Axin1. The effect of TNKS overexpression on Wnt signaling was tested, which revealed that transient transfection of TNKS1 in HEK293 cells dramatically increased STF reporter activity and stabilized β-catenin. This activity requires the Axin binding domain and the SAM domain, but not the PARP domain of TNKS1. It is reported that overexpressed TNKS forms a large lattice-like structure through the SAM domain mediated oligomerization. (De Rycker, M. et al. (2004) Mol Cell Biol 24, 9802-12) We hypothesize that overexpressed TNKS traps Axin in this lattice-like structure and prevents it from performing its normal function in the β-catenin degradation complex.

### Example 5

### Immunoblotting, Immunoprecipitation and GST Pull-Down Assay

Total cell lysates were prepared by cell lysis in RIPA buffer (50 mM Tris-HCl, pH 7.4, 150 mM NaCl, 1% NP-40, 0.5% sodium deoxycholate, 0.1% SDS, 1 mM EDTA). Equal amount of proteins were resolved by SDS-PAGE, transferred to nitrocellulose membranes and probed with indicated antibodies. For co-immunoprecipitation experiments, cells were lysed in EBC buffer (50 mM Tris-HCl, pH 7.4, 150 mM NaCl, 0.5% NP-40, 1 mM EDTA), cleared cell lysates were incubated with indicated antibodies and Protein G-sepharose beads overnight at 4° C. The beads were washed five times with lysis buffer. The bound proteins were dissolved in SDS sample buffer, resolved by SDS-PAGE, and blotted with indicated antibodies. To detect protein ubiquitination and PARsylation in vivo, cells were lysed with RIPA buffer supplemented with 5 mM NEM and 5 μM ADP-HPD to block the activities of Deubiquitinase and PARG, and followed by immunoprecipitation with indicated antibodies.

For the GST pulldown assay, GST-Axin1 fusion proteins were produced in Escherichia coli and purified using glutathione-agarose beads (Amersham Biosciences). HEK293 cells overexpressing Flag-TNKS1 were lysed with EBC buffer, cleared lysates were incubated glutathione-agarose beads charged with GST fusion proteins for, four hours at 4° C., and beads were washed five times with EBC buffer. Bound materials were resolved by SDS-PAGE and blotted with indicated antibodies. To generate cytosolic lysates, cells were scraped into hypotonic buffer (10 mM Tris-HCl pH7.5, 10 mM KCl), and cell lysates were cleared by centrifugation after four freeze-thaw cycles. In all above experiments, 1× protease inhibitor cocktail (Sigma) and 1× phosphatase inhibitor cocktail (Upstate) were added into lysis buffers. Commercial antibodies used in this study include goat anti-Axin1 antibodies (R&D Systems), rabbit anti-Axin2 antibodies and rabbit anti-phospho-β-catenin (pSer33/37/Thr41) antibodies (Cell Signaling Technology), mouse anti-TNKS antibodies (Abeam), mouse anti-HA (HA.11) antibodies (Covance), mouse anti-β-catenin antibodies, rabbit anti-Poly(ADP-ribose) antibodies, and rabbit anti-PARP1 antibodies (BD Pharmingen), mouse anti-ubiquitin antibody (MBL), rabbit anti-GFP antibodies (Clontech), mouse anti-tubulin and mouse anti-Flag (M2) antibodies (Sigma).

### Example 6

### XAV939 Stabilizes Axin by Modulating Ubiquitination and PARsylation of Axin

The increase in Axin protein levels in response to XAV939 treatment could be due to modulation of translation or protein stability. Consistent with the latter possibility, XAV939 treatment was found to significantly prolong the half-life of endogenous Axin2 in SW480 cells. The degradation of Axin is likely mediated by the ubiquitin-proteasome pathway, because the poly-ubiquitination of Axin1 increased significantly after addition of the proteasome inhibitior MG132. In contrast, co-treatment of XAV939 with MG132 significantly diminished Axin1 and Axin2 poly-ubiquitination, suggesting that XAV939 may stabilize Axin by preventing its poly-ubiquitination.

Auto-PARsylation of TNKS or TNKS-mediated TRF1 PARsylation leads to increased ubiquitination and degradation of TNKS or TRF1, respectively. (Yeh, T. Y., et al. (2006) Biochem J 399, 415-25; Chang, W., et al. (2003) Genes Dev 17, 1328-33) Together with internal findings that TNKS physically associates with Axin and requires its PARsylation activity for the regulation of Axin protein levels, this suggested that Axin degradation may be facilitated through direct PARsylation by TNKS. Indeed, TNKS2 was able to PARsylate an Axin1 fragment (a.a. 1-280) containing the TBD in vitro, which was completely inhibited by XAV939 treatment. Using an antibody that specifically recognizes PAR modification, exogenously expressed GFP-Axin was found to be PARsylated in cells. Additionally, the PARsylation signal was strongly reduced in the presence of XAV939, suggesting that Axin PARsylation may be mediated by TNKS in vivo.

Cells were treated with XAV939 to increase endogenous Axin2 levels, which made endogenous Axin2 ubiquitination and PARsylation easier to detect. Axin2 was rapidly degraded within one hour after XAV939 was washed off. As expected, treatment of cells with MG132 blocked Axin2 degradation and strongly increased its poly-ubiquitination. Co-treatment with XAV939 and MG132, however, completely blocked the ubiquitination of Axin2. Interestingly, the anti-PAR antibody reactive signal that co-migrated with Axin2 was detected when cells were treated with MG132 alone but disappeared when cells were also treated with XAV939. Together, these findings suggest that TNKS promotes the ubiquitination and degradation of Axin, which may be mediated, at least in part, through the direct PARsylation of Axin.

### Example 7

### XAV939 Inhibits Colony Formation of APC-Mutant DLD-1 Cancer Cells

Strong genetic evidence linking APC mutant colorectal cancer to constitutively active β-catenin signaling has prompted many efforts to identify WNT pathway inhibitors, but finding pharmacological inhibitors that specifically impede dysregulated WNT pathway activity has proven challenging. Based on internal findings that XAV939 was able to inhibit β-catenin signaling even in APC mutant cells, this compound was tested for its ability to inhibit the proliferation of APC-mutant colorectal cancer cells. When screening a panel of cell lines with inducible shRNAs targeting β-catenin, the colorectal cancer cell line DLD-1 was found to be most sensitive to shRNA-mediated β-catenin inhibition. In addition, the regulation of β-catenin target genes by XAV939 was more robust in DLD-1 compared to SW480 cells. The RKO colorectal cancer cell line was used, which does not harbor any WNT pathway mutations and is insensitive to β-catenin depletion as negative control. Under low serum growth conditions, XAV939 significantly inhibited colony formation of DLD-1 cells, whereas the inactive structural analogue LDW643 did not affect proliferation even at the highest concentration. Importantly, XAV939 did not affect colony formation in the β-catenin independent RKO cells.

The colony formation assay was performed as follows: DLD1 and RKO cells were seeded in low serum growth medium (0.5% FBS) at 500 and 1000 cells/well, respectively, into E-well plates. Sixteen hours after plating, compounds were added at the indicated concentrations. Medium was replenished every two days until colony formation was observed. Colonies were stained by a solution of 2 mg/ml crystal violet in buffered formalin and imaged on a Molecular Imager ChemiDoc XRS System (BioRad).

TNKS1/TNKS2 have been described to regulate mitotic progression, telomere maintenance, and GLUT4 transport. (Canudas, S., et al. (2007) Embo J 26, 4867-78 (2007); Seimiya, H., et al. (2002) J Biol Chem 277, 14116-26) In particular, TNKS1 was proposed to be required for the resolution of sister telomere association or assembly of bipolar spindles, and TNKS1 knockdown was reported to cause strong mitotic arrest. (Chang, P., et al. (2005) Nat Cell Biol 7, 1133-9; Dynek, J. N., et al. (2004) Science 304, 97-100) However, using XAV939 treatment or individual/combinatorial TNKS1/TNKS2 siRNA knockdown did not result in any overt mitotic arrest phenotype with cell lines used in this study at either low or high serum conditions, demonstrating that XAV939 does not inhibit the proliferation of DLD1 cells through an anti-mitotic function.

If the anti-proliferative effect of XAV9393 on DLD1 cells were instead mediated by an increase in Axin protein levels, knockdown of Axin1/2 expression would be predicted to rescue the anti-proliferative effects of compound treatment. Indeed, siRNA-mediated depletion of Axin1/2 completely abolished the anti-proliferative effect of XAV939. Together, these findings indicate that the anti-proliferative effects of XAV9393 in DLD1 cells are due to an Axin-dependent inhibition of WNT pathway signaling.

The cell culture methods described in at least Example 6 were performed as follows: HEK293, SW480, DLD1, and RKO cells were grown in DMEM or RPMI1640 supplemented with 10% FCS in a 37° C. humidified incubator containing 5% CO2. Plasmid transfection was done using Fugene 6 (Roche) and siRNA transfection was done using Darmafect 1 (Dhamacon) according to the manufacturers' instructions.

### Example 8

### Materials and Methods

Any materials and methods used to perform the experiments, and to achieve the results, referred to herein, but not already described in detail, are as follows:

### Example 8a

### Quantitative RT-PCR

Total RNA from compound or siRNA treated cells was extracted using the RNeasy Mini Kit (Qiagen) and reverse transcribed with Taqman Reverse Transcription Reagents (Applied Biosystems) according to the manufacturer's instructions. Transcript levels were assessed using the ABI PRISM 7900HT Sequence Detection System. Real-time PCR was performed in 12 μl reactions consisting of 0.6 μl of 20× Assay-on-Demand mix (premixed concentration of 18 μM for each primer and 5 μM probe), 6 μl Taqman Universal PCR Master Mix, and 5.4 μl cDNA template. The thermocycling conditions utilized were 2 min at 50° C., 10 min at 95° C., followed by 40 cycles of 15 sec at 95° C. and 1 min at 60° C. All experiments were performed in triplicate. Gene expression analysis was performed using the comparative C_{T} method with the housekeeping gene, GUSB, for normalization.

### Example 8B

### Pulse-Chase Experiment

SW480 cells were seeded the day before metabolic labeling at 2 million cells/plate in 10 cm plate. Next day, cells were washed 3× with PBS and starved with DMEM without L-Methionine (Mediatech) for 1 hour followed by labeling with ³⁵S-Methionine (100 μCi/ml) (Amersham) for 30 minutes. After completion of labeling, medium was removed and replaced with medium containing 100× excess of cold Methionine. Cells lysates were harvested by RIPA at the indicated time points. Equal amount of radiolabeled lysates were immunoprecipitated by anti-Axin2 antibody overnight. Immunoprecipitants were washed thoroughly with RIPA buffer the next day before SDS-PAGE and followed by transfer. The radioactive signal was detected by PhosphoImager.

### Example 8C

### Compound Affinity Purification

Compound coupling and affinity purification was essentially performed as described (Bantscheff et al 2007.) A derivatized, bioactive analog LDW639 with a 1° amine group was synthesized to allow coupling onto NHS-activated Sepharose 4 beads (Amersham). 293T cells were homogenized in lysis buffer (50 mM Tris/HCl pH 7.5, 5% glycerol, 1.5 mM MgCl₂, 150 mM NaCl, 20 mM NaF, 1 mM Na₃VO₄, 1 mM DTT, 5 □M calyculin A, 0.8% Igepal-CA630 and a protease inhibitor cocktail) using a Dounce homogenizer on ice. Lysate was pre-cleared by centrifugation and protein concentration measured by Bradford assay. Compounds X and Y were dissolved in dimethyl sulfoxide (DMSO) and added in a final concentration of 20 μM (or DMSO alone) to the lysate for 30 min at 4° C. Then 100 μl of negative control-matrix was added and incubation resumed at 4° C. for another 60 min. After centrifugation, beads were transferred into a column (MoBiTech) and washed. Bound material was eluted with NuPAGE LDS sample buffer (Invitrogen) and eluates were reduced, alkylated, separated on 4-12% NuPAGE gels (Invitrogen), and stained with colloidal Coomassie.

### Example 8D

### Drosophila Reporter Assays

S2R cells were seeded in 384-well plates and treated with indicated dsRNAs for 3 days. Cells were then transfected using Effectene (Qiagen) with 0.5 ng pPac-Renilla, together with 2.5 ng Lef-Luc and 2.5 ng pPac-Lef1 for Wnt reporter assay, 5 ng pcopHSP-BRE-Luc for BMP reporter assay, or 18 ng Draf-Luc for JAK/STAT assay. PUC19 was added as carrier DNA to make up 25 ng of DNA in each well. Twenty-four hours after transfection, 12.5% Wingless conditioned medium, 50 ng/ml recombinant human BMP-2 (R&D Systems), or 50% UPD conditioned medium was added, and luciferase assays were carried out 48 hours later using the Duo-Glo luciferase assay kit (Promega). PCR fragments amplified from S2R cell RNA using T7-linked primers (for White, forward 5′-ACCTGTGGACGCCAAGG-3′ (SEQ ID NO:); reverse, 5′-AAAAGAAGTCGACGGCTTC-3′ (SEQ ID NO:). For TNKS, forward, 5′-GATAGGATTGCGGATGAGGA-3′ (SEQ ID NO:); reverse, 5′-TCCAATGAAGAAGAATCGGG-3′) (SEQ ID NO:) were used for dsRNA production using MEGAscript High yield transcription kit (Ambion). To test the effect of TNKS depletion on Axin, S2R cells stably tranfected with DAxin-3xHA were seeded in 24-well plates and treated with indicated dsRNAs for 5 days.

What is claimed is:

1. A method to treat, prevent, or ameliorate a Wnt signaling-related disorder, comprising administering to a subject in need thereof an effective amount of an agent that modulates the catalytic activity of Tankyrase (TNKS).

2. The method of claim 1, wherein said agent reduces or abrogates the catalytic activity of Tankyrase (TNKS).

3. The method of claim 2, wherein said agent is an inhibitory nucleic acid.

4. The method of claim 2, wherein said agent is a fusion protein.

5. The method of claim 2, wherein said agent is a compound of the invention.

6. The method of claim 2, wherein the Wnt signaling-related disorder is associated with aberrant upregulation of Wnt signaling.

7. The method of claim 6, wherein the Wnt signaling-related disorder is selected from cancer, osteoarthritis, and polycystic kidney disease.

8. The method of claim 7, wherein the Wnt signaling-related disorder is colon cancer.

9-10. (canceled)

11. The method of claim 1, wherein said agent enhances the catalytic activity of Tankyrase (TNKS).

12. The method of claim 11, wherein the Wnt signaling-related disorder is associated with aberrant downregulation of Wnt signaling.

13. The method of claim 12, wherein the Wnt signaling-related disorder is selected from osteoporosis, obesity, diabetes, and neuronal degenerative disease.

14-20. (canceled)

21. A method of identifying an agent capable of modulating Wnt pathway signal transduction, comprising:
a) contacting a biological sample in which the Wnt signaling pathway is active in the presence and absence of a test agent under conditions permitting Wnt signaling and in which Axin protein or stability levels can be measured; and
b) measuring the levels of Axin protein or stability in both the presence and absence of said test agent,
wherein (i) a decrease in Axin protein levels or stability in the presence of the test agent, relative to the absence of the test agent, identifies the test agent as an agonist of Wnt pathway signal transduction, and wherein (ii) an increase in Axin protein levels or stability in the presence of the test agent, relative to the absence of the test agent, identifies the test agent as an antagonist of Wnt pathway signal transduction.

22. The method of claim 21, wherein an increase in Axin protein levels or stability is measured by a decrease in total β-catenin levels, an increase in phospho-β-catenin levels, an increase in Axin protein levels, or increased formation of the Axin-GSK3 complex.

23. The method of claim 21, wherein a decrease in Axin protein levels or stability is measured by an increase in total β-catenin levels, a decrease in phospho-β-catenin levels, a decrease in Axin protein levels, or decreased formation of the Axin-GSK3 complex.

24. The method of claim 21, in which said agent is a small molecule.

25. The method of claim 21, in which said agent is an inhibitory nucleic acid.

26. The method of claim 21, in which said agent is a fusion protein.

27. A method of identifying an agent useful for the treatment of Wnt signaling-related disorders, comprising:
a) contacting a biological sample in which the Wnt signaling pathway is active in the presence and absence of a test agent under conditions permitting Wnt signaling and in which Axin protein levels or stability can be measured; and
b) measuring the levels of Axin protein or stability in both the presence and absence of said test agent,
wherein (i) a decrease in Axin protein or stability levels in the presence of the test agent, relative to the absence of the test agent, identifies the test agent as useful for treating disorders associated with aberrant downregulation of Wnt signaling, and wherein (ii) an increase in Axin protein levels in the presence of the test agent, relative to the absence of the test agent, identifies the test agent as useful for treating disorders associated with aberrant upregulation of Wnt signaling.

28. The method of claim 27, wherein an increase in Axin protein levels or stability is measured by a decrease in total β-catenin levels, an increase in phospho-β-catenin levels, an increase in Axin protein levels, or increased formation of the Axin-GSK3 complex.

29. The method of claim 27, wherein a decrease in Axin protein levels or stability is measured by an increase in total β-catenin levels, a decrease in phospho-β-catenin levels, a decrease in Axin protein levels, or decreased formation of the Axin-GSK3 complex.

30. The method of claim 27, in which said agent is a small molecule.

31. The method of claim 27, in which said agent is an inhibitory nucleic acid.

32. The method of claim 27, in which said agent is a fusion protein.

33-38. (canceled)

39. A method for identifying agents useful for the treatment of Wnt signaling-related disorders, comprising contacting a cell in which the Wnt signaling pathway is active with a test agent and detecting a change in Axin protein levels or stability.

40. The method of claim 39, wherein an increase in Axin protein levels or stability is measured by a decrease in total β-catenin levels, an increase in phospho-β-catenin levels, an increase in Axin protein levels, or increased formation of the Axin-GSK3 complex.

41. The method of claim 39, wherein a decrease in Axin protein levels or stability is measured by an increase in total β-catenin levels, a decrease in phospho-β-catenin levels, a decrease in Axin protein levels, or decreased formation of the Axin-GSK3 complex.

42. A method for inhibiting growth or for inducing apoptosis in a tumor cell of a tumor cell, comprising administering to a subject in need thereof an effective amount of an agent that reduces or abrogates the catalytic activity of Tankyrase (TNKS).

43. The method of claim 42, wherein said agent is an inhibitory nucleic acid.

44. The method of claim 42, wherein said agent is a fusion protein.

45. The method of claim 42, wherein said agent is a compound of the invention.

46-49. (canceled)
